(19) United States
(12) Patent Application Publication
(10) Pub. No.: US 2022/0233756 A1
(43) Pub. Date: Jul. 28, 2022

(54) METHODS AND SYSTEMS FOR TREATING MICROBIAL DISEASE

(71) Applicants: George A. Herzlinger, Belmont, MA (US); Regina E. Herzlinger, Belmont, MA (US)

(72) Inventors: George A. Herzlinger, Belmont, MA (US); Regina E. Herzlinger, Belmont, MA (US)

(21) Appl. No.: 17/611,784

(22) PCT Filed: May 15, 2020
(86) PCT No.: PCT/US20/33210

§ 371 Date: Nov. 16, 2021

Related U.S. Application Data
(60) Provisional application No. 62/947,480, filed on Dec. 12, 2019.
(60) Provisional application No. 62/879,338, filed on Jul. 26, 2019.
(60) Provisional application No. 62/849,683, filed on May 17, 2019.

(51) Int. Cl. A61M 1/36; A61M 1/16; A61M 1/28; B01D 69/08; B01D 71/06

(52) U.S. Cl. CPC A61M 1/362; A61M 1/3638; A61M 1/1672; A61M 1/282; A61M 2205/3313; B01D 71/06; A61M 2202/203; A61M 2202/07; B01D 69/08

(57) ABSTRACT

The present disclosure provides methods and systems for treating a biological fluid of a subject suffering from a microbial infection (e.g., a drug-resistant microbial infection). In some embodiments, these methods and systems involve a complement receptor immobilized on, or otherwise associated with a polymer substrate, for example, high surface area particles, membranes, hollow fibers, and/or other porous or non-porous media. In other embodiments, the methods and systems involve a complement receptor present in a dialysate used in a dialyzer for extracting pathogens out of a biological fluid, for example, the blood of a patient.

### CROSS REFERENCE TO RELATED APPLICATIONS

The present application claims priority to U.S. provisional application Ser. No. 62/849,683, filed May 17, 2019, U.S. provisional application Ser. No. 62/879,338, filed Jul. 26, 2019, and U.S. provisional application Ser. No. 62/947,480, filed Dec. 12, 2019, which applications are hereby incorporated by reference in their entirety.

### BACKGROUND

Antibiotics and other drugs have been used for many years to treat infectious diseases caused by microbes such as bacteria and viruses. However, because of the widespread use of these drugs over time, some pathogens have developed resistance to them, making such drugs less effective.

Drug-resistant infection is a particular problem in hospitals, nursing homes, and other inpatient care facilities where there is a greater prevalence of drug-resistant bacteria and a population of individuals who are ill or otherwise have compromised immune systems. According to the Centers for Disease Control and Prevention (CDC), at least two million people in the United States each year become infected with bacteria that are resistant to antibiotics, and at least 23,000 people die each year as a result of these infections. Examples of antibiotic-resistant infections include Methicillin-Resistant Staphylococcus Aureus (MRSA), Streptococcus pneumoniae, Carbapenem-Resistant Enterobacteriaceae, Clostridium difficile, Drug-Resistant Neisseria gonorrhoeae, Drug-Resistant Malaria, and Multi-drug resistant (MDR) or extensively drug resistant (XDR) Tuberculosis.

There is a critical need for a new way to treat these drug-resistant infections.

### SUMMARY OF THE INVENTION

The present disclosure provides methods and systems for treating a biological fluid of a subject suffering from a microbial infection (e.g., a drug-resistant microbial infection). In some embodiments, these methods and systems involve a complement receptor immobilized on, or otherwise associated with a polymer substrate, for example, high surface area particles, membranes, hollow fibers, and/or other porous or non-porous media. In other embodiments, the methods and systems involve a complement receptor present in a dialysate used in a dialyzer for extracting pathogens out of a biological fluid, for example, the blood of a patient.

The complement system is part of the human immune system and aids in clearing pathogens and other harmful microbes from the body. In the body, complement fragments (or ligands) attach to certain cell surfaces (e.g. pathogens), thereby tagging them and attracting complement receptors to them. Phagocytes, which are cells that protect the body, have complement receptors which recognize the complement fragments that are bound to the pathogens. The phagocytes then ingest the tagged pathogens (via phagocytosis) allowing the immune system to remove the tagged pathogens.

The present disclosure includes embodiments that bring complement receptors, e.g., CRIg (Complement Receptor of the Immunoglobulin superfamily), into fluid contact with the blood (or other biological fluid) of a subject, in an effort to filter pathogens from the blood, thereby preventing and/or diminishing the growth and/or spread of bacteria or other infections in the subject. When pathogens are opsonized with complement proteins including components of C3 such as iC3b (alternatively spelled C3bi), they can be detected by various receptors including, for example, CR1, CR3, CR4, and CRIg. As opsonized pathogens are slower to be removed from the body than non-opsonized pathogens, extracorporeal devices that can quickly remove opsonized pathogens would aid in the removal of these pathogens from the subjects. Both gram positive and gram negative bacteria can be opsonized, including, but not limited to, Staphylococcus Aureus and Escherichia Coli. Other bacteria which can be opsonized and can bind to the receptors include, for example, Streptococcus pneumonia, Haemophilus influenza type b, Streptococcus pneumonia type 3, and Streptococcus pyogenes. Viruses (such as e.g., HIV, influenza, or coronavirus) can also be opsonized with iC3b. Fungi such as Candida albicans and candida stellatoidea can be opsonized as well.

It has been found that certain biocompatible polymers will activate component C3, a protein found in human blood. These findings are described in “Quantitative Measurement of C3 Activation at Polymer Surfaces,” Blood, Vol. 57, No. 4 (April), 1981, which is incorporated by reference herein, and is attached hereto as Appendix A. In a separate study, a group from the National Institute of Health showed that a compound called thymic stromal lymphopoietin (TSLP) in the skin can enhance killing of bacteria by human neutrophils (white blood cells), in part, by interaction with the complement system (West et al., “TSLP acts on neutrophils to drive complement-mediated killing of methicillin-resistant Staphylococcus aureus,” J. Immunology, May 1, 2016, 196). Their data indicate that TSLP may augment innate immune cells and complement to fight bacterial infections. It was found that TSLP mediates its anti-bacterial effect by engaging C5, a complement protein which is part of a cascade of complement proteins: C6, C7, C8, C9 that lead to formation of a “membrane attack complex” which can effectively destroy bacteria.

Polymer is used in certain embodiments, e.g., in the form of high surface area beads, as a substrate on which a complement receptor, e.g., CRIg, is bound or otherwise associated, to facilitate fluid contact of the complement receptor with components of the blood, including the pathogen. Other embodiments use another form of polymer, e.g., a membrane, hollow fibers, or other porous or non-porous solid. Moreover, in certain embodiments, Factor H is conjugated to the polymer in addition to the pathogen binding receptor to inhibit excess complement activation. However, under certain conditions, complement activation might be helpful to opsonize bacteria so that the receptors can pull pathogens out of the blood. Excessive complement activation may lead to serious adverse events, so a careful balance of activation and restriction of the complement pathway may be necessary. Therefore, in certain embodiments, the beads or other form of polymer are conjugated with Factor H, in addition to complement receptors such as CRIg. In certain embodiments, the pathogen binding receptor(s) is/are selected from CR1, CR3, CR4, and/or CRIg and are conjugated to the surface of the polymer directly through covalent bonding, electrostatically, or by using some other linkers or extenders (for example, His-Tag, biotin/streptavidin, neutravidin, Concanavalin, avidin, Amines, or the like).

In addition to the methods and systems described above which use a high surface area polymer with one or more complement receptors immobilized on, or otherwise associated with, the polymer, alternative embodiments involve a complement receptor present in a dialysate used in a dialyzer for extracting pathogens from a biological fluid (e.g., the blood of a patient). The dialysate may also contain Factor H, particularly if the dialysis system uses a polymeric membrane that may lead to excessive complement activation. In these systems, the dialysate carrying the complement receptor and/or Factor H is brought into fluid contact with components of the blood, particularly pathogens. The polymeric membrane may have a pore size that allows passage of pathogens from the blood on one side of the membrane into the dialysate on the other side of the membrane while disallowing passage of blood cells into the dialysate. In these embodiments, the complement receptor and/or Factor H may be in soluble form. Pathogens are thereby removed from the blood of the patient which is returned to the patient.

The present disclosure provides, among other things, systems for removing microbes from a biological fluid of a subject. In some embodiments, such systems include: at least one filter cartridge comprising a plurality of beads with at least one complement receptor attached thereto or otherwise associated therewith; and at least one tubing line for conducting the biological fluid from the subject to the at least one filter cartridge (e.g., and, in certain embodiments, back to the subject). In some embodiments, the at least one complement receptor comprises at least one of CR1, CR2, CR3, CR4, CR3aR, CR5aR, C1qR, C1qRp, CRIg, and a complement binding portion of any thereof.

In certain embodiments, at least one bead of the plurality of beads has attached thereto (or otherwise associated therewith) at least one complement inhibitor selected from the group consisting of sialic acid, Factor H protein (or a fragment thereof), a mini fH protein, and a fH-recruiting peptide. In some embodiments, said plurality of beads comprises a high surface area polymer.

In some embodiments, systems provided herein for removing microbes from a biological fluid of a subject include: at least one filter cartridge having a chamber comprising at least one porous membrane with at least one complement receptor disposed thereon or therewithin, attached thereto, or otherwise associated therewith; and at least one tubing line for conducting the biological fluid from the subject to the at least one filter cartridge (e.g., and, in certain embodiments, back to the subject). In some embodiments, the at least one complement receptor comprises at least one of CR1, CR2, CR3, CR4, CR3aR, CR5aR, C1qR, C1qRp, CRIg, and a complement binding portion of any thereof.

In certain embodiments, at least one porous membrane has disposed thereon or therewithin at least one complement inhibitor selected from the group consisting of sialic acid, Factor H protein (or a fragment thereof), a mini fH protein, and a fH-recruiting peptide. In some embodiments, the at least one porous membrane comprises a high surface area polymer.

In certain embodiments, the polymer has a coating (e.g., at least a partial coating) on its surface. In some embodiments a coating is or comprises sialic acid, fH protein (or a fragment thereof), a mini fH protein, or a fH-recruiting peptide.

In certain embodiments, the polymer has a coating (e.g., at least a partial coating) on its surface that selectively binds the one or more complement receptors from the biological fluid onto the surface of the polymer (e.g., wherein the method comprises conducting a flow of the biological fluid over the surface of the polymer to concentrate the one or more complement receptors onto the polymer, whereupon the polymer activates or enhances activation of the one or more complement receptors, thereby enhancing the killing of bacteria in the biological fluid by innate immune cells (e.g., neutrophils) in the biological fluid).

In some embodiments, the present disclosure provides a bead for use in a system for removing microbes from a biological fluid of a subject, said bead having at least one complement receptor attached thereto or otherwise associated therewith. In some embodiments, the at least one complement receptor comprises at least one of CR1, CR2, CR3, CR4, CR3aR, CR5aR, C1qR, C1qRp, CRIg, and a complement binding portion of any thereof. In some embodiments, the bead comprises a diameter from about 1 micron to about 1000 microns. In some embodiments, the bead has attached thereto or associated therewith at least one complement inhibitor selected from the group consisting of sialic acid, Factor H protein (or a fragment thereof), a mini fH protein, and a fH-recruiting peptide. In some embodiments, the bead comprises a high surface area polymer.

In some embodiments, the present disclosure provides filter cartridges for use in a system for removing microbes from a biological fluid of a subject. In some embodiments, said filter cartridges include at least one bead, the at least one bead comprising at least one complement receptor. In some embodiments, the at least one complement receptor comprises at least one of CR1, CR2, CR3, CR4, CR3aR, CR5aR, C1qR, C1qRp, CRIg, and a complement binding portion of any thereof. In some embodiments, the at least one bead has attached thereto or associated therewith at least one complement inhibitor selected from the group consisting of sialic acid, Factor H protein (or a fragment thereof), a mini fH protein, and a fH-recruiting peptide. In some embodiments, the at least one bead comprises a high surface area polymer.

The present disclosure further provides methods for treating a biological fluid of a subject having a microbial disease (e.g., extracorporeal treatment). In some embodiments, said methods include: contacting the biological fluid of the subject with a high surface area polymer having a substance immobilized thereupon or associated therewith, said substance comprising one or more complement receptors.

In some embodiments, said methods include: contacting the biological fluid of the subject with a high surface area polymer having a substance immobilized thereupon or associated therewith, said substance comprising one or more complement components, thereby treating the microbial disease.

In some embodiments, one or more complement receptors comprises at least one of CR1, CR2, CR3, CR4, CR3aR, CR5aR, C1qR, C1qRp, CRIg, and a complement binding portion of any thereof. In some embodiments, the high surface area polymer has attached thereto or associated therewith at least one complement inhibitor selected from the group consisting of sialic acid, Factor H protein (or a fragment thereof), a mini fH protein, and a fH-recruiting peptide. In certain embodiments, the complement receptor is CRIg or a complement-binding portion thereof. In certain embodiments, the substance immobilized on the high surface area polymer comprises CRIg and a complement inhibitor selected from: sialic acid, Factor H protein (or a fragment thereof), a mini fH protein, or a fH-recruiting peptide.

In some embodiments, one or more complement components comprise one or more members selected from the group consisting of Complement C1, Complement C2, Complement C3, Complement C4, Complement C5, Complement C6, Complement C7, Complement C8, Complement C9, a fragment of any one of the preceding, a convertase of any one of the preceding (e.g., C3 convertase, C5 convertase), and any other protein or protein fragment in a complement cascade [e.g., (i) a covalently bound fragment of C3 or C4 (e.g. C3b, C4b), e.g., responsible for oponization; (ii) an anaphylatoxin (e.g., C5a, C3a) or anaphylatoxin receptor on a leukocyte (e.g., C5aR, C3aR), e.g., responsible for chemotaxis and activation of leukocytes; (iii) a membrane-attack complex (e.g., C5b-9), e.g., responsible for lysis of bacteria and/or cells; (iv) C3b or C4b, or a fragment of either, bound to an immune complex or antigen, or a CR1-4 receptor on a B cell or APC, e.g., responsible for augmentation of antibody response; (v) any of C3a, C5a, C3aR, or C5aR on a T cell or APC, e.g., responsible for enhancement of T-cell response to APC and/or reduction of Treg function; and (vi) any of C1q, a covalently bound fragment of C3 (e.g., C3b) or C4 (e.g., C4b), or CR1 on an erythrocyte, or a CR1-4 receptor on a phagocyte, e.g., responsible for clearance of immune complexes from tissues and/or clearance of apoptotic cells]. In some embodiments, one or more complement components comprise consisting of C1 inhibitor (C1-INH), MCP, DAF, CR1, C4 binding protein (C4BP), Factor H, thrombomodulin, Factor I, CD59, S-protein (vitronectin), and clusterin (SP-40, 40). In some embodiments, one or more complement components comprise CR1, CR2, CR3, CR4, CR3aR, CR5aR, C1qR, C1qRp, CRIg, and a complement binding portion of any thereof.

In some embodiments, the method further includes administering at least one drug to the subject. In some embodiments, the at least one drug to be administered in accordance with the provided methods is an antibiotic. Exemplary antibiotics include tetracyclines, aminoglycosides, macrolides, clindamycin, linezolid (oxazolidinone), chloramphenicol, streptograrnins, quinilones, sulfonamides, trimethoprim, rifampin, vancomycin, trimethoprim/sulfamethoxazole, doxycycline, ceftobiprole, ceftaroline, clindamycin, dalbavancin, Daptomycin, fusidic acid, mupirocin, omadacycline, oritavancin, tedizolid, telavancin, and tigecycline.

In some embodiments, the biological fluid is a member selected from the group consisting of blood, serum, plasma, cerebrospinal fluid, lymph, synovial fluid, and amniotic fluid. In certain embodiments, the biological fluid is blood, serum or plasma.

In some embodiments, the high surface area polymer has surface area of at least 50,000 cm²/g (e.g., more preferably, at least 100,000 cm²/g, more preferably, at least 150,000 cm²/g, or more preferably, at least 200,000 cm²/g). In some embodiments, the high surface area polymer comprises particles having an average dimension (e.g., diameter) of less than 1 micrometer. In some embodiments, the polymer comprises one or more members selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof. In certain embodiments, the high surface area polymer is or comprises nylon (e.g., Nylon-6, 6). In certain embodiments, the high surface area polymer is or comprises poly (methyl methacrylate) (PMMA).

In some embodiments, the microbial disease to be treated in a subject is or comprises a bacterial, viral, fungal, and/or protozoan infection. In some embodiments, the biological fluid comprises bacteria, virus, fungus, and/or protozoa, such as Methicillin-Resistant Staphylococcus Aureus, Streptococcus pneumoniae, Carbapenem-Resistant Enterobacteriaceae, Clostridium difficile, Drug-Resistant Neisseria Gonorrhoeae, Drug-Resistant Malaria, Tuberculosis (e.g., MDR or XDR tuberculosis), coronavirus (e.g., SARS-CoV-2), or an infectious Candida species.

In some embodiments, the subject has an antibiotic-resistant infection (e.g., Methicillin-Resistant Staphylococcus Aureus, Streptococcus Pneumoniae, Carbapenem-Resistant Enterobacteriaceae, Clostridium Difficile, Drug-Resistant Neisseria Gonorrhoeae, Drug-Resistant Malaria, Multi-drug resistant (MDR) or extensively drug resistant (XDR) Tuberculosis), and wherein the contacting step treats the subject of the infection (e.g., reducing the severity of the infection or eliminating the infection). In some embodiments, the biological fluid comprises methicillin-resistant Staphylococcus aureus (MRSA), and wherein the contacting step enhances attachment of the at least one complement receptor to at least one pathogen in the biological fluid.

In some embodiments, the substance further comprises thymic stromal lymphopoietin (TSLP) (e.g., wherein the TSLP enhances the ability of innate immune cells in the biological fluid (e.g., neutrophils) and the complement receptor to kill bacteria in the biological fluid). In some embodiments, the substance further comprises a drug or other agent that enhances the ability of innate immune cells in the biological fluid (e.g., neutrophils) and complement to kill bacteria in the biological fluid.

In some embodiments, the contacting step comprises conducting the biological fluid of the subject through a filter (e.g., a membrane or other structure) comprising the high surface area polymer.

In some embodiments, provided methods further include returning the biological fluid to the subject following the contacting step.

The present disclosure also provides systems for treating a biological fluid of a subject having a microbial disease (e.g., infection or other disease caused by a pathogenic organism, e.g., virus, bacteria, fungus, or protozoa). In some embodiments, said systems include: (i) a pump, (ii) a tubing line fluidly connected to the pump, and (iii) a treatment filter, where the pump conducts the biological fluid from the subject to the treatment filter (e.g., and, in certain embodiments, back to the subject), and where the treatment filter comprises a high surface area polymer having a substance immobilized thereupon or associated therewith, said substance comprising one or more complement receptors.

In some embodiments, the system further includes an air trap and/or air detector through which the tubing line passes prior to return of the biological fluid to the subject. In some embodiments, the pump removes bubbles and/or microbubbles in the biological fluid before the biological fluid passes through the treatment filter. In some embodiments, the biological fluid is a member selected from the group consisting of blood, serum, plasma, cerebrospinal fluid, lymph, synovial fluid, and amniotic fluid.

In some embodiments, the substance immobilized on the high surface area polymer comprises a complement receptor (e.g., CR1, CR2, CR3, CR4, CR3aR, CR5aR, C1qR, C1qRp, CRIg, or a complement binding portion of any thereof). In certain embodiments, the substance immobilized on the high surface area polymer comprises CRIg and a complement inhibitor selected from: sialic acid, Factor H protein (or a fragment thereof), a mini fH protein, or a fH-recruiting peptide.

In some embodiments, the high surface area polymer has surface area of at least 50,000 cm²/g (e.g., more preferably, at least 100,000 cm²/g, more preferably, at least 150,000 cm²/g, or more preferably, at least 200,000 cm²/g). In some embodiments, the high surface area polymer is a membrane (e.g., hollow fiber membrane or other porous or nonporous membrane). In some embodiments, the high surface area polymer comprises particles having an average dimension (e.g., diameter) of less than 1 micrometer. In some embodiments, the high surface area polymer comprises poly(methyl methacrylate) (PMMA). In some embodiments, the high surface area polymer comprises nylon (e.g., Nylon-6, 6). In some embodiments, the high surface area polymer comprises one or more members selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof.

In some embodiments, a biological fluid to be treated with a system of the present invention is from a subject with a bacterial, viral, fungal, and/or protozoan infection. In some embodiments, the biological fluid comprises bacteria, virus, fungus, and/or protozoa, such as Methicillin-Resistant Staphylococcus Aureus, Streptococcus pneumoniae, Carbapenem-Resistant Enterobacteriaceae, Clostridium difficile, Drug-Resistant Neisseria gonorrhoeae, Drug-Resistant Malaria, Tuberculosis (e.g., MDR or XDR tuberculosis), coronavirus (e.g., SARS-CoV-2), or an infectious Candida species.

In some embodiments, the biological fluid comprises methicillin-resistant Staphylococcus aureus (MRSA), and wherein the system enhances attachment of the one or more complement receptors to at least one pathogen in the biological fluid. In certain embodiments, the biological fluid comprises one or more infectious microbes selected from the group consisting of Methicillin-Resistant Staphylococcus Aureus, Streptococcus Pneumoniae, Carbapenem-Resistant Enterobacteriaceae, Clostridium Difficile, Drug-Resistant Neisseria Gonorrhoeae, Drug-Resistant Malaria, and Multi-drug resistant (MDR) or extensively drug resistant (XDR) Tuberculosis, and wherein the system enhances the killing of the one or more infectious microbes. In certain embodiments, the substance further comprises thymic stromal lymphopoietin (TSLP) (e.g., wherein the TSLP enhances the ability of innate immune cells in the biological fluid (e.g., neutrophils) and the complement to kill bacteria in the biological fluid).

In some embodiments, provided are methods of operating systems of the present disclosure, said methods comprising conducting a flow of the biological fluid over the surface of the high surface area polymer in the treatment filter, whereupon the polymer activates or enhances activation of the one or more complement receptors, thereby enhancing the in the biological fluid attachment of the one or more complement receptors onto at least one pathogen in the biological fluid.

The present disclosure further provides methods for treating a biological fluid of a subject having a microbial disease comprising: contacting the biological fluid of the subject with particles (and/or fibers or other substrate) associated with a complement receptor, wherein the particles (and/or fibers or other substrate) bind microbes in the biological fluid.

Also provided herein are methods for removing microbes from a biological fluid of a subject, comprising: contacting the biological fluid of the subject with particles (and/or fibers or other substrate) associated with a complement receptor, wherein the particles (and/or fibers or other substrate) bind microbes in the biological fluid.

In some embodiments, the complement receptor is a CR1, CR2, CR3, CR4, CR3aR, CR5aR, C1qR, C1qRp, CRIg, or complement binding portion of any thereof. In certain embodiments, the complement receptor is a CR1 or complement binding portion thereof and/or a CR2 or complement binding portion thereof. In other embodiments, the complement receptor is a CRIg or complement binding portion thereof.

In some embodiments, the particles are further associated with a complement inhibitor selected from the group consisting of sialic acid, Factor H protein (or a fragment thereof), a mini fH protein, and a fH-recruiting peptide. In some embodiments, the particles (and/or fibers or other substrate) associated with the complement receptor comprise a high surface area polymer selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof. In some embodiments, the particles associated with the complement receptor comprise magnetic beads. In some embodiments, the particles are contained within a replaceable cartridge.

In some embodiments, provided herein are systems for treating a biological fluid of a subject comprising: (i) a pump, (ii) a tubing line fluidly connected to the pump, and (iii) a cartridge, wherein the pump conducts the biological fluid through the tubing line from the subject to the cartridge (e.g., and, in certain embodiments, back to the subject), and wherein the cartridge comprises particles (and/or fibers or other substrate) associated with one or more complement receptors, wherein the particles (and/or fibers or other substrate) of the cartridge is/are capable of binding microbes in the biological fluid.

In some embodiments, provided herein are systems for removing microbes from a biological fluid of a subject, comprising: a pump and a tubing line for conducting the biological fluid from the subject to a cartridge (e.g., and, in certain embodiments, back to the subject); and the cartridge comprising particles (and/or fibers or other substrate) associated with one or more complement receptors, wherein the particles (and/or fibers or other substrate) of the cartridge are capable of binding microbes in the biological fluid.

In some embodiments, such systems include one or more complement receptors that comprise at least one member selected from the group consisting of CR1, CR2, CR3, CR4, CR3aR, CR5aR, CRIg, C1qR, C1qRp, and a complement binding portion of any thereof. In some embodiments, the one or more complement receptors comprises (i) a CR1 or complement binding portion thereof and/or (ii) a CR2 or complement binding portion thereof. In certain embodiments, the one or more complement receptors comprises CRIg or a complement binding portion thereof.

In some embodiments, the particles are further associated with a complement inhibitor selected from the group consisting of sialic acid, Factor H protein (or a fragment thereof), a mini fH protein, or a fH-recruiting peptide. In some embodiments, the particles (and/or fibers or other substrate) associated with the one or more complement receptors comprise a high surface area polymer selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof. In some embodiments, the particles associated with the one or more complement receptors comprise magnetic beads. In some embodiments, the cartridge is a replaceable cartridge. In some embodiments, the cartridge is constructed such that cells and/or other substances >5 μm, >4 μm, >3 μm, >2 μm or >1 μm in characteristic dimension (e.g., diameter or length) are unable to enter the cartridge (and/or a treatment portion thereof).

In some embodiments, provided herein are systems for treating a biological fluid of a subject comprising: (i) at least one dialyzer, (ii) a first tubing line for conducting the biological fluid from the subject to the at least one dialyzer; and (iii) a second tubing line for conducting a dialysate to the at least one dialyzer, wherein the dialysate comprises a soluble complement receptor.

In some embodiments, the soluble complement receptor comprises at least one of CR1, CR2, CR3, CR4, CR3aR, CR5aR, C1qR, C1qRp, CRIg, and a complement binding portion of any thereof. In some embodiments, the soluble complement receptor comprises CRIg or a complement binding portion thereof.

In some embodiments, the dialysate further comprises at least one complement inhibitor selected from the group consisting of sialic acid, Factor H protein (or a fragment thereof), a mini fH protein, and a fH-recruiting peptide. In some embodiments, the dialyzer comprises at least one membrane (e.g., a membrane comprising a high surface area polymer).

In some embodiments, such a system further comprises a dialysate supply tank fluidly connected to the second tubing line; and a compressed air tank fluidly connected upstream of the dialysate supply tank.

In some embodiments, such a system may further comprise a dialysate inlet through which dialysate enters the dialyzer; a dialysate exit, through which dialysate exits the dialyzer; and at least one sensor disposed within at least one of the dialysate inlet and the dialysate exit. In some embodiments, the at least one sensor comprises a spectrographic UV sensor.

In some embodiments, such a system may include a dialyzer that further includes a blood inlet, through which the biological fluid enters the dialyzer; a top header, the top header in fluid communication with and downstream of the blood inlet; a blood exit, through which the biological fluid exits the dialyzer; a bottom header, the bottom header in fluid communication with and upstream of the blood exit; and at least one hollow fiber membrane disposed between the top header and the bottom header, wherein the at one hollow fiber membrane fluidly connects the top header and the bottom header.

In some embodiments, the dialyzer further comprises at least one hollow fiber membrane. In some embodiments, the at least one hollow fiber membrane further comprises a high surface area polymer.

In some embodiments, the high surface area polymer comprises one or more members selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof. In some embodiments, the high surface area polymer has surface area of at least 50,000 cm²/g (e.g., more preferably, at least 100,000 cm²/g, more preferably, at least 150,000 cm²/g, or more preferably, at least 200,000 cm²/g).

In some embodiments, the at least one hollow fiber membrane has a cylindrical shape comprising an interior and an exterior, wherein the biological fluid flows through the interior of the hollow fiber membrane, and wherein the dialysate flows around the exterior of the hollow fiber membrane. In some embodiments, the at least one hollow fiber membrane fluidly connects the at least one biological fluid to the dialysate.

In some embodiments, the dialyzer comprises a membrane between the biological fluid and the dialysate, the membrane having a structure (e.g., pores) such that cells and/or other substances >5 μm, >4 μm, >3 μm, >2 μm, or >1 μm in a characteristic dimension (e.g., diameter or length) are unable to pass from the biological fluid into the dialysate (e.g., but pathogens can pass from the biological fluid into the dialysate).

In some embodiments, the biological fluid is a member selected from the group consisting of blood, serum, plasma, cerebrospinal fluid, lymph, synovial fluid, and amniotic fluid.

The present disclosure further provides methods for treating a biological fluid of a subject having a microbial disease (e.g., extracorporeal treatment), comprising: (i) providing a dialysate comprising at least one complement receptor on a first side of a membrane (e.g., a porous polymer membrane, e.g., a hollow fiber membrane); and (ii) providing the biological fluid on a second side of the membrane, wherein the membrane is structured (e.g., has pores) to allow passage of a pathogen from the biological fluid through the membrane into the dialysate.

In some embodiments, said methods further include returning the biological fluid to the subject following a period of time in which components of the biological fluid are in fluid contact with the dialysate. In some embodiments, the dialysate flows on the first side of the membrane. In some embodiments, the biological fluid flows on the second side of the membrane.

The present disclosure also provides methods for treating a biological fluid of a subject having a microbial infection, the method comprising: (i) introducing a dialysate to a peritoneal cavity of the subject, wherein the dialysate comprises at least one complement receptor, such that the dialysate is in contact with the biological fluid; and (ii) removing the dialysate from the biological fluid after sufficient time to opsonize a pathogen. In some embodiments, said method is or includes peritoneal dialysis, such as continuous ambulatory peritoneal dialysis (CAPD), continuous cycling peritoneal dialysis (CCPD), and/or intermittent peritoneal dialysis (IPD). In some embodiments, the dialysate comprises one or more components for opsonization of the pathogen, e.g., as detailed herein.

Throughout the description, where systems or compositions are described as having, including, or comprising specific components, or where methods are described as having, including, or comprising specific steps, it is contemplated that, additionally, there are systems or compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The following description is for illustration and exemplification of the disclosure only, and is not intended to limit the invention to the specific embodiments described.

The mention herein of any publication, for example, in the Background section, is not an admission that the publication serves as prior art with respect to any of the claims presented herein. The Background section is presented for purposes of clarity and is not meant as a description of prior art with respect to any claim.

### BRIEF DESCRIPTION OF THE DRAWINGS

The Drawings included herein, which is composed of the following Figures, is for illustration purposes only and not for limitation.

FIG. 1 depicts an exemplary schematic of a system for treating a biological fluid of a subject that includes a treatment filter, a pump and a tubing line for conducting the biological fluid from the subject to a treatment filter. In certain embodiments, as depicted a system can include means for returning the treated biological fluid back to the subject. The schematic depicted is adapted from a description of hemodialysis by the National Institute of Health, https://www.niddk.nih.gov/health-information/kidney-disease/kidney-failure/hemodialysis.

FIG. 2 depicts a schematic of a system for treating a biological fluid, according to certain embodiments of the present disclosure.

FIG. 3 depicts a schematic of a system for treating a biological fluid, according to certain embodiments of the present disclosure.

FIG. 4 depicts a schematic of a system for treating a biological fluid, according to certain embodiments of the present disclosure.

FIG. 5 depicts a filter cartridge according to certain embodiments of the present disclosure.

FIG. 6 depicts a portion of a filter cartridge according to certain embodiments of the present disclosure.

FIG. 7 depicts a portion of a filter cartridge according to certain embodiments of the present disclosure.

FIG. 8 depicts a filter cartridge according to certain embodiments of the present disclosure.

FIG. 9 depicts a filter cartridge according to certain embodiments of the present disclosure.

FIG. 10 depicts a filter cartridge according to certain embodiments of the present disclosure.

FIG. 11 depicts a filter cartridge according to certain embodiments of the present disclosure.

FIG. 12 depicts a filter cartridge according to certain embodiments of the present disclosure.

FIG. 13 depicts a filter cartridge according to certain embodiments of the present disclosure.

FIG. 14 depicts a filter cartridge according to certain embodiments of the present disclosure.

FIG. 15 depicts a filter cartridge according to certain embodiments of the present disclosure.

FIG. 16 depicts a filter cartridge according to certain embodiments of the present disclosure.

FIG. 17 depicts a dialyzer according to certain embodiments of the present disclosure.

FIG. 18 depicts a peritoneal dialysis system according to certain embodiments of the present disclosure.

### DESCRIPTION OF THE INVENTION

The present disclosure provides methods and systems for treating a biological fluid of a subject suffering from a microbial infection (e.g., a drug-resistant microbial infection). These methods and systems involve complement receptors immobilized on, or otherwise associated with, high surface area particles (e.g., high surface area polymer particles).

In one aspect, the invention is directed to a method for treating a biological fluid of a subject having a microbial disease (e.g., infection or other disease caused by a pathogenic organism, e.g., virus, bacteria, fungus, or protozoa), the method comprising: contacting the biological fluid of the subject with a high surface area polymer having a substance immobilized thereupon or associated therewith, said substance comprising one or more complement receptors (e.g., wherein the high surface area polymer is at least partially coated with, or otherwise physically and/or chemically attached to, the substance comprising the one or more complement receptors), thereby treating the microbial disease.

In certain embodiments, the biological fluid is a member selected from the group consisting of blood (e.g., unmodified whole blood or modified blood), serum, plasma, cerebrospinal fluid, lymph, synovial fluid, and amniotic fluid.

For example, in some embodiments, a system and method for clearing MRSA and/or other pathogens from the blood of a subject are provided. Complement receptors are attached to beads or other substrate contained in a cartridge that is part of a pump. The MRSA in the blood attaches to the beads. The cartridges are discarded and replaced (or recycled) until the blood is cleared of MRSA. The pump may include a mechanism for discarding and replacing the cartridge. The system may include a sensor/detector for determining whether sufficient clearance of the MRSA (or other invasive species) from the blood. In certain embodiments, the cartridge contains magnetic beads. In certain embodiments, the cartridge (or a treatment portion thereof) does not admit blood cells, which are returned to the subject. The beads immobilize the MRSA, E coli, and/or other antigens, toxins, or other debris with complement attached. For size comparison, red blood cells are about 7.5 micrometers in diameter, white blood cells are about 15 micrometers in diameter, MRSA is about 1 micron, and E. coli is about 2.0 micrometers long and 0.25 to 1.0 micrometer in diameter. Blood of the subject is drawn through the cartridge containing the complement receptor coated beads. In certain embodiments, complement is added to the blood in the cartridge. Variables of the system whose values (or ranges) may be optimized for a particular usage include, for example, (i) the flow rate of the biological fluid through the cartridge (and/or residence time therein), (ii) the temperature at one or more locations along the flow path, and (iii) the species, concentration, and/or source of complement to be added to the blood (if needed).

### Systems

In another aspect, the invention is directed to a system for treating a biological fluid of a subject having a microbial disease (e.g., infection or other disease caused by a pathogenic organism, e.g., virus, bacteria, fungus, or protozoa), the system comprising: a pump and a tubing line for conducting the biological fluid from the subject to a treatment filter (e.g., and, in certain embodiments, back to the subject); and the treatment filter, wherein the treatment filter comprises a high surface area polymer having a substance immobilized thereupon or associated therewith, said substance comprising one or more complement receptors (e.g., wherein the high surface area polymer is at least partially coated with, or otherwise physically and/or chemically attached to, the substance comprising the one or more complement receptors).

In certain embodiments, the system further comprises an air trap and/or air detector through which the tubing line passes prior to return of the biological fluid to the subject.

In certain embodiments, the pump is configured to remove bubbles and/or microbubbles in the biological fluid before it passes through the treatment filter.

In certain embodiments, the biological fluid is a member selected from the group consisting of blood (e.g., unmodified whole blood or modified blood), serum, plasma, cerebrospinal fluid, lymph, synovial fluid, and amniotic fluid).

In some embodiments, systems of the present disclosure for treating a biological fluid comprise a filter and/or cartridge comprising particles. In some embodiments, particles comprise polymeric and/or magnetic material. In some embodiments, a system comprises a filter and/or cartridge comprising high surface area polymer particles. In some embodiments a filter and/or cartridge comprises high surface area polymer particles associated with one or more complement receptors. In some embodiments, a system comprises a filter and/or cartridge comprising particles that are or include magnetic beads. In some embodiments a filter and/or cartridge comprises magnetic beads associated with one or more complement receptors.

In some embodiments, blood cells are unable to enter the cartridge (or chamber or portion thereof). In some embodiments, cells and/or other substances >5 μm, >4 μm, >3 μm, >2 μm, or >1 μm in characteristic dimension (e.g., diameter or length) are unable to enter a cartridge (or chamber or portion thereof) of the present disclosure, while any microbes (e.g., MRSA, E. coli, etc.) pass therein, such that the microbes attach to particles (and/or fibers) in the cartridge, thereby removing a portion (e.g., at least 50%, 75%, 90%, 95%, or 99%) of the MRSA or other undesirable microbes from the cell-free portion of the biological fluid.

In some embodiments, a system of the present disclosure comprises a filter and/or cartridge comprising particles, and components to flow a biological fluid through the filter and/or cartridge. In some embodiments, a system further comprises a pump and tubing. In some embodiments, a system is or comprises a dialysis system.

In some embodiments, a biological fluid in the system has a temperature within a range of 25° C. to 37° C. In some embodiments the temperature is 27° C., 30° C., 33° C., 35° C., or 37° C. In some embodiments, the temperature of a biological fluid in a cartridge comprising complement receptors is within a range of 25° C. to 37° C. In some embodiments the temperature is 27° C., 30° C., 33° C., 35° C., or 37° C. In some embodiments, a system of the present disclosure has a temperature controller (e.g., to control the temperature of the biological fluid at one or more locations in a treatment flow path of a system of the present disclosure).

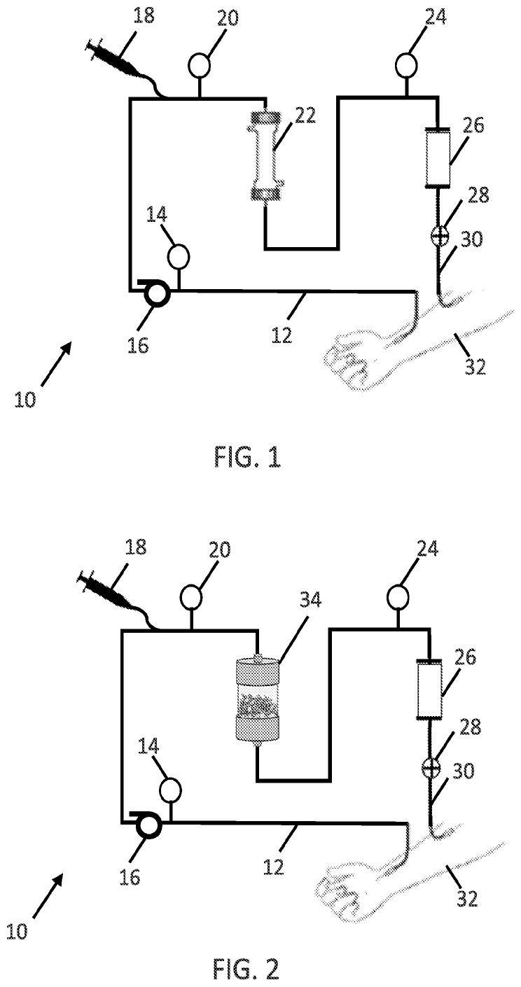

FIG. 1 depicts an illustrative arrangement of system components. In this illustrative embodiment, blood is drawn from the subject 32 by a pump 16 into a tubing line 12. In some embodiments, the pump 16 provides for reduction or elimination of microbubbles that are present in the blood prior to being contacted with the filter 22. Optionally, heparin may be administered in the blood flow line at the heparin pump 18 to prevent clotting. Other anti-coagulants may also be used such as CPDA-1, CPD, ACD, and/or Hirudin. The blood passes through a filter 18 comprising the high surface area polymer, embodiments of which are described herein. Treated blood then exits the filter and passes through an air trap/detector 26 before it is returned to the subject. The system may also include pressure monitors 14, 20, 24 (arterial pressure, inflow pressure, and venous pressure). In certain embodiments, components that come into contact with the biological fluid are biocompatible and/or sterilizable and/or replaceable. In certain embodiments, the pump 16 receives electric power by an Alternating Current (AC) wall outlet. In certain embodiments, the pump 16 is operated from battery power.

In some embodiments, the system pumps fluid (such as blood) at rates within a range bounded by a lower limit and an upper limit, the upper limit being larger than the lower limit. In some embodiments, the lower limit may be about 50 ml/min, about 60 ml/min, about 70 ml/min, about 80 ml/min, about 90 ml/min, about 100 ml/min, about 150 ml/min, about 200 ml/min, about 300 ml/min, about 400 ml/min, about 500 ml/min, about 600 ml/min, about 700 ml/min, about 800 ml/min, about 900 ml/min, about 1000 ml/min, or about 1200 ml/min. In some embodiments, the upper limit may be about 100 ml/min, about 150 ml/min, about 200 ml/min, about 300 ml/min, about 400 ml/min, about 500 ml/min, about 600 ml/min, about 700 ml/min, about 800 ml/min, about 900 ml/min, about 1000 ml/min, about 1200 ml/min, about 1400 ml/min, about 1500 ml/min, about 1600 ml/min, about 1800 ml/min, or about 2000 ml/min.

In certain embodiments, the system pumps fluid at low flow rates, e.g., non-zero flow rates of less than about 100 ml/min, or less than about 50 ml/min, or less than about 40 ml/min, or less than about 30 ml/min, or less than about 20 ml/min, or less than about 15 ml/min, or less than about 10 ml/min.

In some embodiments, the system comprises a cartridge 22 comprising particles and/or fibers associated with one or more complement receptors, and a pump 16 and a tubing line 12 for conducting biological fluid from a subject 32 to the cartridge 22. In some embodiments, pump flows at a rate that is within a range of 10 ml/min to 2000 ml/min. In some embodiments, pump flows at a rate that is within a range of 100 ml/min to 1500 ml/min.

In some embodiments, the residence time of fluid in the cartridge 22 is within a range of 1 second to 10 minutes. In some embodiments, the residence time of fluid in the cartridge 22 is less than 5 minutes, less than one minute, less than 45 seconds, or less than 30 seconds.

Variations of this system may also be implemented. For example, in certain embodiments, the biological fluid is not directly drawn from the subject 32, but is drawn from a plastic blood bag or other biological fluid storage container. In certain embodiments, blood is not drawn by a pump 16 but may travel through the filter 22 via gravity (in which case, in certain embodiments, air bubbles, e.g., microbubbles, may be removed from the biological fluid prior to its passing through the filter via an air bubble removal device other than a pump). In certain embodiments, treated blood is not immediately returned to the subject 32 but is stored, e.g., in a blood storage container. Various other biological fluids may be treated with embodiments of the system described above [e.g., blood (e.g., unmodified whole blood or modified blood), serum, plasma, cerebrospinal fluid, lymph, synovial fluid, and amniotic fluid].

In certain embodiments, the system includes a pump 16 or other device for removal of microbubbles from the biological fluid before the fluid passes through the treatment filter 22. The system may additionally have an air trap 26 and/or air detector 28 through which the treated biological fluid passes before being returned to the subject 32 (or before being stored), but, in certain embodiments, it is important that air bubbles be removed from the blood or other biological fluid prior to being introduced to the high surface area polymer filter. This is because microbubbles (intravascular gas) may be present in tissues during certain infections, and are found to trigger an immune response. The microbubbles are essentially tagged as foreign invaders, to be destroyed by the body's immune system. In certain embodiments, the pump 16 (or other device) eliminates air bubbles present in the biological fluid before the fluid passes into the high surface area polymer filter for treatment. In some embodiments, the high surface area polymer filter 22 is a cartridge.

In some embodiments, a system of the present disclosure comprises a dialysis system comprising a filter 22 or cartridge, wherein the filter 22 or cartridge comprises particles (e.g., high surface area polymer particles and/or magnetic particles) (or fibers) associated with one or more complement receptors.

In some embodiments, a polymer cartridge 22 can be replaced until a biological fluid is substantially free of invasive microbial content. In some embodiments, substantially free of invasive microbial content is >80% of invasive microbial content removed, >85% of invasive microbial content removed, >90% of invasive microbial content removed, >95% of invasive microbial content removed, >96% of invasive microbial content removed, >97% of invasive microbial content removed, >98% of invasive microbial content removed, or >99% of invasive microbial content removed. In some embodiments, a polymer cartridge 22 can be replaced and treatment resumed until a biological fluid being treated by a system of the present disclosure (e.g., a dialysis system) is essentially completely free of invasive microbial content.

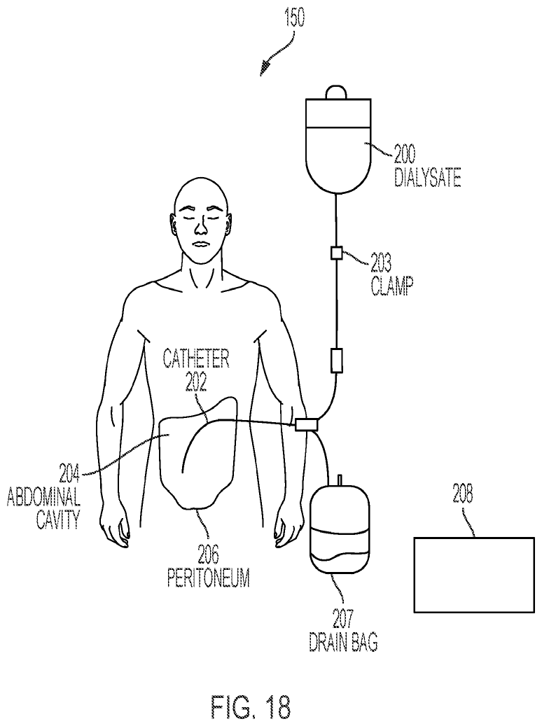

In another aspect, the invention is directed to a peritoneal system for treating a biological fluid of a subject having a microbial disease (e.g., infection or other disease caused by a pathogenic organism, e.g., virus, bacteria, fungus, or protozoa). FIG. 18 depicts an illustrative arrangement of system components for peritoneal dialysis.

### Illustrative System Embodiments

The system of the present disclosure provides equipment for treating a biological fluid of a subject suffering from or at risk for a microbial infection (e.g., a microbial infection in the blood). The system equipment may be used in connection with dialysis equipment and other commonly used medical equipment. The system 10 of FIG. 1 may use a filter 22 or may use a dialyzer (i.e., in place of or in addition to the filter 22) as a mechanism for transferring the complement to the blood.

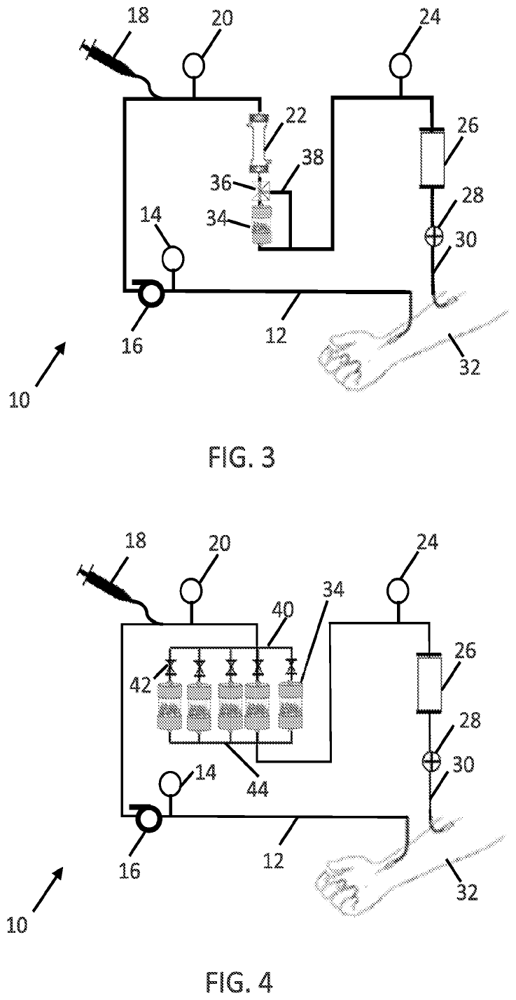

FIG. 2 illustrates an embodiment of the system 10 of the present disclosure including a fluid line 12 from the patient 32, an arterial pressure monitor 14, a pump 16, a heparin pump 18, a dialyzer inflow pressure monitor 18, a filter cartridge 34, a venous pressure monitor 24, an air trap 26, an air detector claim 28, and a return line 30 back to the patient 32. In the embodiment of FIG. 3, the system 10 includes a filter cartridge 34 with beads coated with CRIg complement.

FIG. 3 illustrates an embodiment of the system 10 of the present disclosure including a fluid line 12 from the patient 32, an arterial pressure monitor 14, a pump 16, a heparin pump 18, a dialyzer inflow pressure monitor 18, a dialyzer 22, filter cartridge 34, a venous pressure monitor 24, an air trap 26, an air detector claim 28, and a return line 30 back to the patient 32. In the embodiment of FIG. 4, the system 10 includes both a dialyzer 22 and a filter cartridge 34 with beads coated with CRIg. In the embodiment of FIG. 3, the system 10 also includes a bypass valve 36 and a bypass line 38. Because too high of a pressure drop across the system 10 may result in blood clotting, the pressure upstream and downstream of the dialyzer 22 and filter 34 can be monitored to ensure the pressure drop remains in an acceptable range. If the pressure drop becomes too large, however, the cartridge filter 34 with beads coated with CRIg can be bypassed by directing the flow of blood through the bypass line 38 via the bypass valve 36. In the embodiment of FIG. 3, a patient can simultaneously receive dialysis treatment as well as antibacterial and/or microbial infection treatment (i.e., via the CRIg) thereby reducing the impact on the patient and potentially allowing the patient to receive microbial infection treatment sooner, i.e., while the patient is already receiving dialysis treatment.

FIG. 4 illustrates an embodiment of the system 10 of the present disclosure including a fluid line 12 from the patient 32, an arterial pressure monitor 14, a pump 16, a heparin pump 18, a dialyzer inflow pressure monitor 18, a filter cartridge 34, a venous pressure monitor 24, an air trap 26, an air detector claim 28, and a return line 30 back to the patient 32. In the embodiment of FIG. 4, the system 10 includes multiple filter cartridges 34, allowing blood to be routed through multiple filter cartridges 34 simultaneously (thereby increasing the available specific surface area through which the CRIg may be transferred to the blood). Blood may be routed into an intake manifold 40 from which it can flow into each of the filter cartridges 34 that are in service. One or more filter valves 42 is disposed downstream of the intake manifold 40, but upstream of each filter cartridge 34 allowing each filter 34 to be independently placed into service or taken out of service. An exit manifold 44 is disposed downstream of the plurality of filter cartridges. The exit manifold 44 receives all of the blood that flows through whichever filter cartridges 34 are in service, and routes the blood into a single line through which it can flow to the downstream system components and eventually back to the patient 32. Operating the system with additional filter cartridges 34 in service may result in a slightly increased flow rate through the system and may also increase the residence time that blood remains in each filter cartridge 34. In the embodiment of FIG. 4, the multiple filter cartridges 34 allow one or more filters to be placed in service and/or taken out of service without interrupting the steady flow of blood through the filter cartridge(s) 34 (and back to the patient 32).

In each of the system embodiments of FIGS. 1-4, 1) other components (e.g., components not illustrated in the figures) may also appear in the system, 2) the system may not include every component illustrated in the figures, and 3) the components in the system may be arranged in different configurations. For example, in systems that include both a dialyzer 22 and a filter cartridge 34 with beads coated with CRIg, the dialyzer 22 and filter cartridge 34 may be arranged in a parallel flow configuration with valves upstream of each, allowing either component to be placed in service and/or taken out of service, as the circumstance may dictate. The dialyzer 22 and the filter cartridge 34 with beads coated with CRIg may also be configured in series (similar to FIG. 3) but with a bypass valve upstream of the dialyzer 22 with an associated bypass line allowing blood to selectively be routed around the dialyzer (while also allowing for blood to flow through both the dialyzer 22 and filter 34 in series, when both dialysis and filtration are desired). Each of the system embodiments of FIGS. 1-4, may also include a blood flow meter for measuring the flow of blood through the system 10 (which may aid in assessing how long a patient should be treated for and/or remain in treatment). It is also possible to approximate the blood flow through the system using the differential pressure between the various system pressure monitors 14, 20, 24. Other embodiments according to the present disclosure may include multiple filter cartridges 34 in a parallel configuration (similar to FIG. 4) in series with a dialyzer 22 (i.e., either upstream or downstream), a bypass valve 36, a bypass line 38, and/or other system components, as discussed above.

### Filter Cartridge

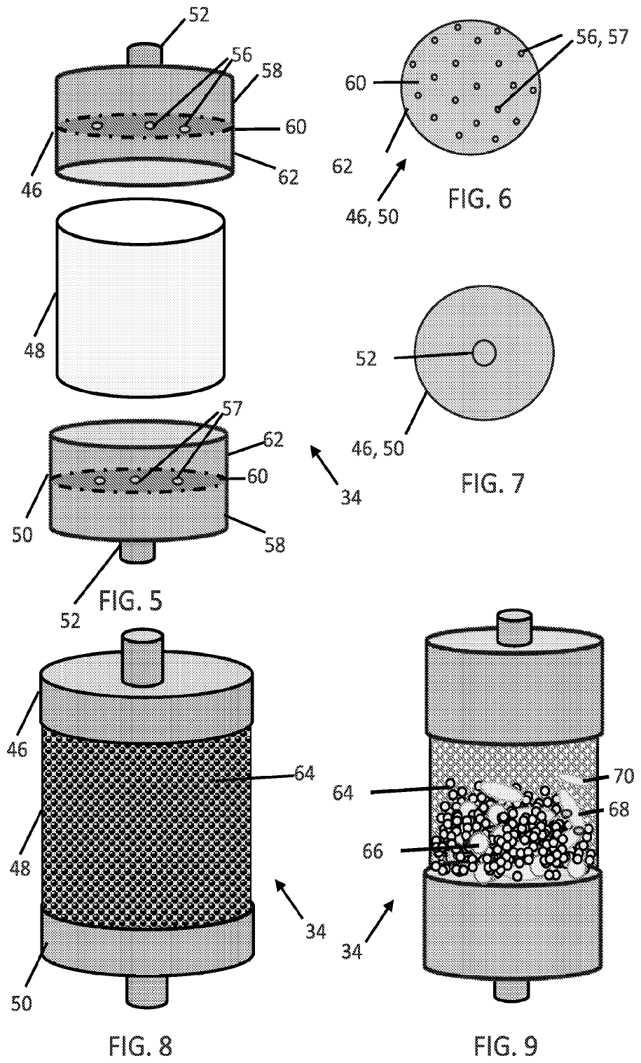

FIGS. 5-16 illustrate embodiments of a filter cartridge 34 of the present embodiments. FIG. 5 illustrates a filter cartridge 34 which includes a top portion 46, a center portion 48, and a bottom portion 50. In the embodiment of FIG. 5, each of the top, center, and bottom portions 46, 48, 50 are shown as individual components, however, when in service, the filter cartridge 34 will operate as a single component with the center portion 48 being attached to both the bottom portion 50 and the top portion 46. Each of the top and bottom portions 46, 50 may include an adaptor 52 to which fluid lines may be connected and through which blood may flow into and out of the filter cartridge 34.

Referring still to FIG. 5, each of the top and bottom portions 46, 50 may include a header portion 58, a filter plate 60, and a lip portion 62. Blood that enters the top portion 46 enters the header portion 58 first, and then flows into the center portion 48 through a plurality of top holes 56 which are distributed around the filter plate 60 and help to spread blood to the full cross-sectional area of the center portion 48. The top lip portion 62 may be disposed around the top of the center portion 48 when the filter cartridge 34 is assembled and/or fabricated such that the lip portion overlaps with the center portion 48 when assembled. The lip portions 62 and the top and bottoms of the center portion 48 may all be threaded such that the lip portions 62 may be screwed onto the center portion (or vice versa), creating a tight seal with the center portion 48. In other embodiments, the center portion 48 may slide into the lip portions 62 (i.e., the lip portions of both the top and bottom portions 46, 50) and may be sealed tightly via compression fit, epoxy, adhesion, tape, glue, fusion, and other suitable mechanisms.

Still referring to FIG. 5, after flowing into the center portion 48, blood flows past and around beads coated with CRIg (shown in FIGS. 8-11, 13 and 14) such that the CRIg can attach to and/or attract pathogens in the blood. Blood can then flow through the filter plate 60 in the bottom portion 50 and into the header portion 58 of the bottom portion 50 where it collects prior to flowing out of the filter cartridge 34 via the adaptor 52 disposed in the bottom portion 50. The bottom holes 57 (i.e., in the bottom portion 50) may be sized such that the beads (not shown) cannot fit through them, while still allowing blood to flow through. Stated otherwise, the diameter of the bottom holes 57 needs to be smaller than the diameter of the smallest bead (not shown). For example, in one embodiment the bottom holes 57 in the bottom portion 50 may be from about 10 microns to about 40 microns in diameter while the beads may have diameters from about 40 microns and to about 500 microns. In another embodiment, the bottom holes 57 in the bottom portion 50 may be from about 25 microns to about 120 microns in diameter while the beads may have diameters from about 120 microns and to about 1000 microns. In another embodiment, the holes 57 in the bottom portion 50 may be from about 15 microns to about 150 microns in diameter while the beads may have diameters from about 160 microns and to about 1000 microns. The filter cartridge 34 illustrated in FIG. 5 may include a generally cylindrical shape (i.e., circular cross-section). In other embodiments, the filter cartridge 34 may have a square, triangular, and/or other shaped cross-section. The filter cartridge 34 may be composed of any suitable material such as polymer, thermoplastic, glass, metal, ceramic, composite materials, other materials and combinations thereof. The filter cartridge 34 may also be composed (or partially composed) of a transparent material such as glass or transparent polymers such that the interior of the filter cartridge 34 is visible externally.

FIG. 6 illustrates a bottom (under-side) view of the top and/or bottom portions 46, 50, including pluralities of top holes and/or bottom holes 56, 57 disposed therein. The top and/or bottom portions 46, 50 may include a greater number of holes 56, 57 disposed therein than what is shown in FIG. 6. For example, in some embodiments top and/or bottom portions 46, 50 may include as many as 20, 50, 100, 500, 1,000, 5000, 10,000, 20,000, 50,000 and/or higher numbers of holes disposed therein. The lip 62 and the filter plate 60 are also visible in the embodiment depicted in FIG. 6.

FIG. 7 illustrates a top view of the top and/or bottom portions 46, 50, including the adaptor 52 disposed thereon.

FIG. 8 illustrates a perspective view of a fully-assembled filter cartridge 34 including the top portion 46, the center portion 48 and the bottom portion 50. In the embodiment of FIG. 8, beads 64 coated with CRIg are packed into the interior of the filter cartridge 34. The beads 64 illustrated in FIG. 8 may include a spherical shape and/or an ellipsoid shape, which has a higher specific surface area (i.e., surface area per unit volume) than spheres. In other embodiments, the beads 64 may be spherical, ellipsoid, cubic, prism-shaped, and/or other shapes. In addition, the beads 64 may include a mix of shapes and sizes, and may be tightly packed, as illustrated in FIG. 8.

FIG. 9 illustrates a perspective view of a fully-assembled filter cartridge 34 including beads coated with CRIg contained in the interior of the filter cartridge 34. In the embodiment of FIG. 9, the beads may include large ellipsoid beads 68, large spherical beads 66, small spherical beads 64, and/or small ellipsoid beads 70. The smaller beads 64, 70 may be used to fill up the spaces between the bigger beads 68, 66, thereby increasing the total specific surface area of CRIg-coated beads within the filter cartridge 34. In addition, the beads 64, 66, 68, 70 may include holes and passageways (not shown) disposed therein to further increase the specific surface area. The beads 64, 66, 68, 70 may also include a rough surface finish, again to increase the specific surface area.

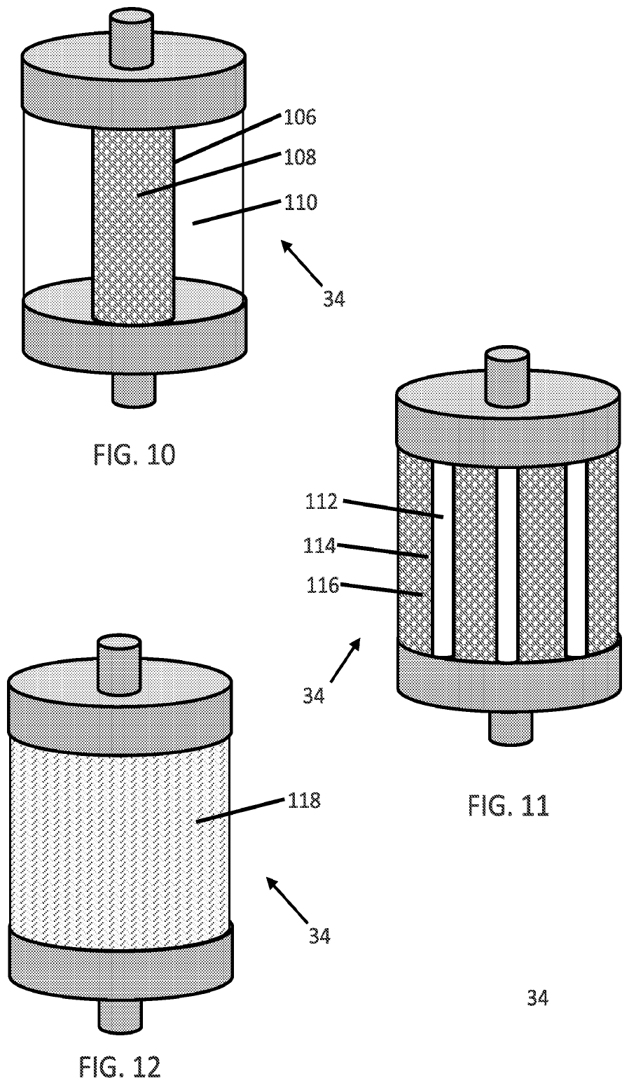

FIG. 10 illustrates a filter cartridge 34 according to the present disclosure including a bead-filled center column 108 which includes a semi-permeable membrane wall 106 through which CRIg may be transferred to the blood or through which pathogens in the blood may travel to reach the CRIg. The beads in the center column 108 are coated with CRIg. An annulus 110 surrounds the center column 108 and allows blood to flow through the filter cartridge (i.e., from the top portion to the bottom portion around the outside of the center column 108) with CRIg attaching to pathogens as the blood flows past.

FIG. 11 illustrates a filter cartridge 34 according to the present disclosure including multiple blood tubes 112 through which blood flows. The surrounding area 116 (i.e., surrounding the blood tubes 112 within the filter cartridge 34) includes beads 64, 66, 68, 70 coated with CRIg. The blood tubes 112 include a semi-permeable wall 114 allowing the CRIg to transfer therethrough such that it may attach to the pathogens in the blood within the blood tubes 112. The pathogens in the blood may also travel through the semi-permeable wall 114 to the surrounding areas 116, which include CRIg-coated beads 64, 66, 68, 70. In the embodiments of FIGS. 10 and 11, the filter cartridge 34 may include different numbers and arrangements of the respective center bead column(s) 108 and/or blood tubes 112.

FIG. 12 illustrates a filter cartridge 34 according to the present disclosure including a permeable membrane 118 coated with CRIg allowing pathogens in the blood to attach to the CRIg as the blood flows through the filter cartridge 34. In each of the embodiments of FIGS. 1-11, 13 and 14, the beads 64, 66, 68, 70 coated with CRIg may have diameters from about 1 micron to about 1000 microns while the pores in the membranes as well as the bottom holes 57 (in the bottom portion 50) may have diameters from about 1 micron to about 250 microns.

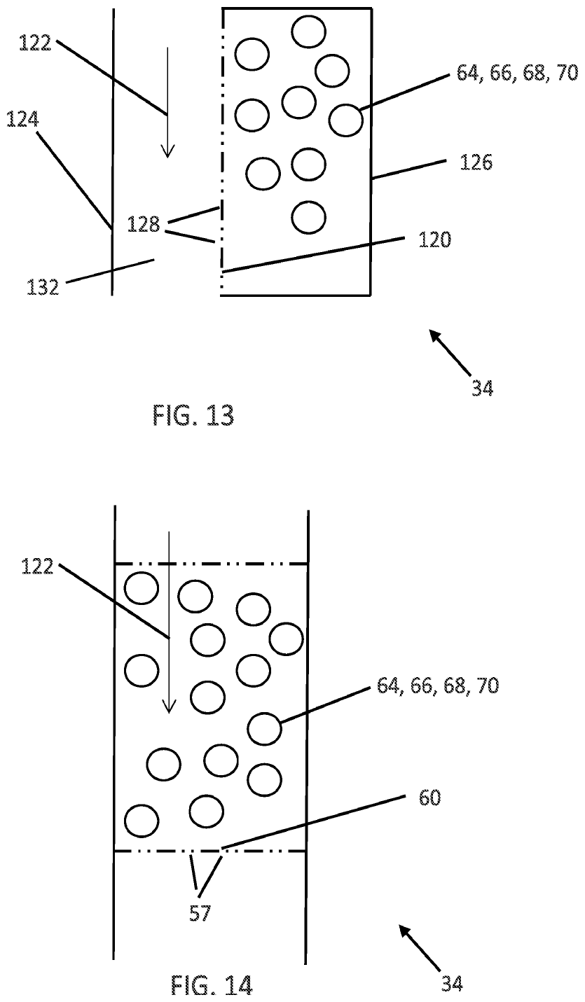

FIG. 13 illustrates a filter cartridge 34 according to the present disclosure including a bead container 126 for holding beads 64, 66, 68, 70 coated with CRIg and/or other complement receptors. The bead container 126 may have a circular cross-section as well as a square, rectangular and/or other suitably-shaped cross-section. One or more walls of the bead container 126 (and/or the cylindrical outer surface of the bead container 126 in embodiments including circular cross-section) and/or portions thereof may include a porous membrane 120 allowing blood pathogens and other potentially hazardous blood components to pass through, while simultaneously permitting red blood cells to flow past. The porous membrane 120 includes pores 128 that are sized such that they are smaller than the minimum diameter of red blood cells.

As the flow of blood 122 passes the porous membrane 120, only the pathogens and other hazardous blood components with diameters smaller than the diameter of the pores 128 can pass through the porous membrane 120, where they are attracted to (and captured by) the beads 64, 66, 68, 70 coated with CRIg and/or other complement receptors within the bead container 126. The embodiment of FIG. 13 may include beads 64, 66, 68, 70 (coated with CRIg and/or other complement receptors) within the bead container 126 that are packed within the bead container 126 much more densely than what is illustrated in FIG. 13.

Still referring to FIG. 13, in some embodiments the porous membrane 120 may include pores 128 that are less than or equal to about 7 microns in diameter. In other embodiments, the porous membrane 120 may include pores 128 that are greater than or equal to about 0.1 microns in diameter and less than or equal to about 6 microns in diameter. In other embodiments, the porous membrane 120 may include pores 128 that are greater than or equal to about 0.1 microns in diameter and less than or equal to about 5 microns in diameter. In other embodiments, the porous membrane 120 may include pores 128 that have diameters that are within various sub-ranges between about 0.1 microns and about 7 microns. In the embodiment of FIG. 13 the beads 64, 66, 68, 70 may include diameters from about 15 microns to about 120 microns, or from about 20 microns to about 100 microns, or from about 30 microns to about 90 microns, or from about 40 microns to about 80 microns, or from about 50 microns to about 70 microns, or from about 55 microns to about 65 microns, as well as all sub-ranges therebetween, as well as diameters from about 120 microns to about 1000 microns. The bead container 126 may be disposed within the interior of the filter cartridge 34 or in other embodiments, the bead container may about the flow of blood 122 within a blood flow passage 132 (for example adjacent to and/or partially forming a wall 124 of the blood flow passage 132), as long as at least one surface of the bead container 126 is adjacent to (and in flow communication with) the blood flow passage 132).

Referring still to FIG. 13, in some embodiments, the bead container 126 may be a standalone container that is placed within the filter cartridge 34 such that blood can flow by while pathogens can be attracted to the beads 64, 66, 68, 70 coated with complement receptors such as CRIg. For example, in one embodiment, the bead container 126 may include a cubic shape with one, two, three, four, five and/or all six of the sides of the cube including the porous membrane 120. In some embodiments, adjacent and/or opposing sides of the cubic bead container 126 may include porous membranes 120. In other embodiments, the bead container 126 may include a cylindrical shape with porous membranes 120 disposed within the top surface, bottom surface, and/or the curved longitudinal surface of the cylinder. In another embodiment, the bead container 126 may include a spherical geometry with one or more porous membranes 120 disposed around the outer surface of the sphere, or portions thereof. In other embodiments, the bead container 126 may include other suitable shapes and arrangements of the porous membrane(s) 120.

FIG. 14 illustrates a filter cartridge 34 according to the present disclosure similar to the embodiments of FIGS. 5-9, and including beads 64, 66, 68, 70 coated with CRIg and/or other complement receptors that attract pathogens in the blood as the flow of blood 122 passes by. The filter cartridge 34 may include a filter plate 60 with holes 57 disposed therein preventing the beads 64, 66, 68, 70 from passing, but allowing blood to flow therethrough. The beads 64, 66, 68, 70 coated with CRIg and/or other complement receptors depicted in FIG. 14 may be packed within the filter cartridge 34 much more densely (or tightly) than what is illustrated in FIG. 14. The holes 57 in the filter plate 60 may be sized such that they are about 10 microns or greater in diameter, allowing blood cells to pass through.

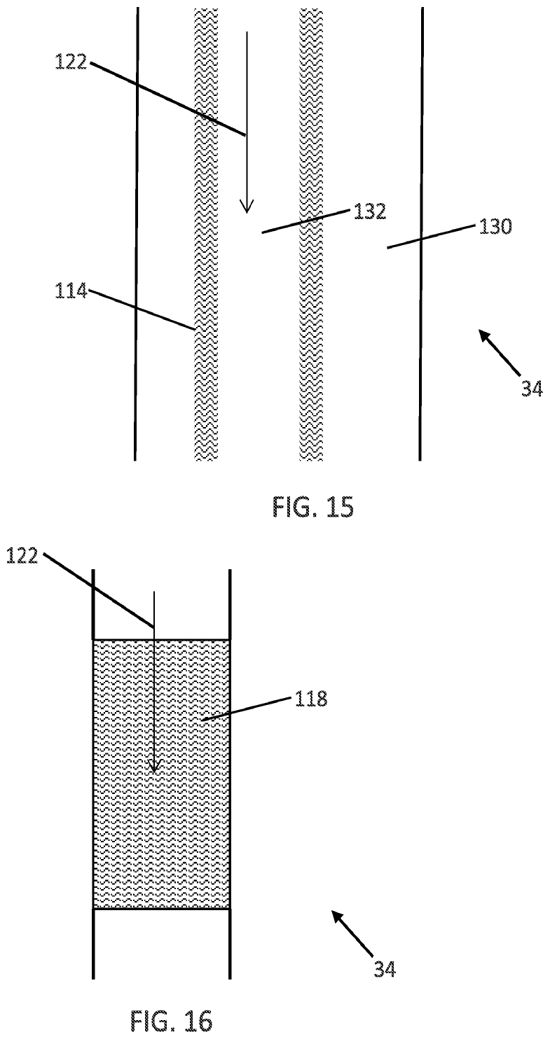

FIG. 15 illustrates a filter cartridge 34 according to the present disclosure including a flow of blood 122 through one or more blood flow passages 132. Each of the one or more blood passages 132 may include a porous membrane wall 114 that is coated with one or more complement receptors (for example CRIg) to capture pathogens form the blood. The porous membrane wall 114 allows pathogens to pass through to an open space 130 surrounding the one or more blood flow passages 132. The open space 130 may contain dialysate or saline fluid in order to flush the pathogens away from the one or more blood flow passages 132. Pores in the porous membrane wall 114 may be sized similar to the pores 128 in the porous membrane 120 of FIG. 15 such that pathogens can pass through, but blood must flow past.

FIG. 16 illustrates a filter cartridge 34 according to the present disclosure similar to the embodiment of FIG. 12, including a porous filter 118 coated with complement receptors (such as CRIg) that capture pathogens as the flow of blood 122 passes through.

In one embodiment, the fully assembled filter cartridge 34 according to the present disclosed embodiments may include a square-shaped cross-sectional area with a width and length of from about 0.5 inches to about 1.2 inches and a height from about 2 inches to about 6 inches. In another embodiment, the fully assembled filter cartridge 34 may include a square-shaped cross-sectional area with a width and length of from about 0.7 inches to about 1.0 inch and a height from about 3 inches to about 5 inches. In another embodiment, the fully assembled filter cartridge 34 may include a square-shaped cross-sectional area with a width and length of about 0.85 inches and a height of about 3.8 inches. In another embodiment, the fully assembled filter cartridge 34 may include a circular cross-sectional area with a diameter of from about 0.8 inches to about 1.2 inches and a height of from about 3.2 inches to about 4.6 inches. In another embodiment, the fully assembled filter cartridge 34 may include a circular cross-sectional area with a diameter of about 1 inch and a height of about 3.8 inches. In another embodiment, the fully assembled filter cartridge 34 may include ellipsoid beads with a diameter from about 50 microns to about 70 microns and a length from about 100 microns to about 150 microns. In another embodiment, the fully assembled filter cartridge 34 may include spherical beads with a diameter from about 50 microns to about 70 microns. In other embodiments, the filter cartridge 34 and beads 64, 66, 68, 70 may be other shapes and sizes.

### Dialyzer

In some embodiments, a dialyzer is for use in hemodialysis.

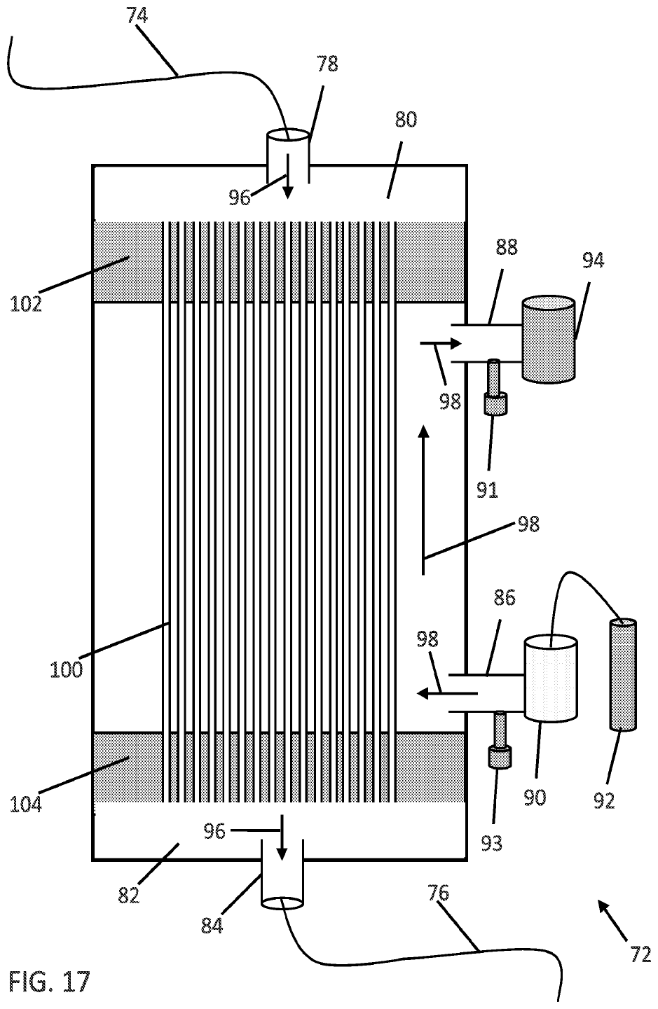

FIG. 17 illustrates an embodiment according to the present disclosure including a dialyzer 72 that may be used to transfer CRIg to the blood. The dialyzer 72 may include an inlet line 74 fluidly coupled to a blood inlet 78 for delivering blood to the dialyzer 72, as well as an outlet line 76 fluidly connected to a blood outlet 84 allowing blood to exit the dialyzer 72. The dialyzer 72 may further include a top header 80, where blood collects when it enters the dialyzer 72, as well as a bottom header 82, where blood collects prior to exiting the dialyzer 72. A plurality of hollow fiber membranes 100 are disposed between the top and bottom headers 80, 82, thereby fluidly connecting the top and bottom headers 80, 82. Each of the hollow fiber membranes may have an inner diameter from about 100 microns to about 300 microns, or from about 150 microns to about 250 microns, or from about 180 microns to about 200 microns. Each of the hollow fiber membranes may also have a wall thickness from about 10 microns to about 60 microns, or from about 20 microns to about 50 microns, or from about 30 microns to about 40 microns. A top support 102 supports the plurality of hollow fiber membranes 100 at the top end while simultaneously acting as the lower boundary of the top header 80. Similarly, a bottom support 104 supports the plurality of hollow fiber membranes 100 at the bottom end while simultaneously acting as the upper boundary of the bottom header 82. The direction of the flow of blood 96 is also illustrated in FIG. 17.

Referring still to FIG. 17, the dialyzer 72 also includes a dialysate supply tank 90 for storing and supplying clean dialysate to the dialyzer 72, as well as a dialysate inlet 86, fluidly connecting the dialysate supply tank 90 to the interior of the dialyzer 72. The dialyzer 72 may also contain a compressed air tank 92 for pressurizing the dialysate supply tank 90, thereby creating dialysate flow 98 through the dialyzer 72. The dialyzer 72 also may include a dialysate exit 98 fluidly connected to a dialysate waste tank 94 for storing the waste dialysate. Dialyzer 72 employs a dialysate that may include a soluble form of CRIg (for example, a human CRIg variant in solution form). As CRIg soluble dialysate flows through the dialyzer 72, CRIg attaches to and/or attracts pathogens in the blood via pores in the plurality of hollow fiber membranes 100. As such, the pores in the hollow fiber membranes 100 may be sized and shaped to enhance attachment of the CRIg to pathogens in the blood, and vice versa. In addition to soluble CRIg, the dialysate may also include complement inhibitor such as sialic acid, Factor H protein (or a fragment thereof), a mini fH protein, or a fH-recruiting peptide.

Still referring to FIG. 17, the dialyzer 72 may also include a first spectrographic ultraviolet (UV) sensor 91 disposed in the dialysate exit 88 and a second spectrographic UV sensor 93 disposed at the dialysate inlet 86. The first and second spectrographic UV sensors 91, 93 may be used to monitor the amount of soluble CRIg flowing into and out of the dialyzer 72. For example, if more CRIg is flowing into the dialyzer 72 than the amount of CRIg flowing out of the dialyzer 72, it can be inferred that some of the CRIg flowing into the dialyzer 72 is attaching to pathogens in the blood via the pores in the hollow fiber membranes. Similarly, if about the same amount of soluble CRIg is flowing both into and out of the dialyzer, it can be inferred that not much of the CRIg is attaching to pathogens in the blood. In this scenario, the pressure in the dialysate supply tank 90 can be increased via the compressed air tank 92 in order to increase the flow of dialysate through the dialyzer 72 to encourage increased CRIg attachment. The first and/or second spectrographic UV sensors 91, 93 may also be used in connection with the filter cartridge 34 of FIGS. 5-16, as well as with the systems of FIGS. 1-4. As such, by using the first and/or second spectrographic UV sensors 91, 93 to assess the amount of CRIg flowing through the blood downstream of the filter cartridge 34, it is possible to derive an attachment rate (or an eradication rate). When the attachment rate and/or the eradication rate begins to drop, it may indicate that a filter cartridge 34 should be taken out of service and/or replaced with a new filter cartridge 34 (for example, by opening and/or closing valves 42 upstream of each filter cartridge 34, as illustrated in FIG. 5). The dialyzer 72 of FIG. 17 may have a cross-section that is circular, rectangular, square, and/or other suitable shapes.

The porous filter/porous membrane 118, porous walls 106, 114, 128 filter plate 60, filter cartridge 34, hollow fiber membranes 100, and/or beads 64, 66, 68, 79 of FIGS. 1-17 may be constructed of a material which induces complement activation or minimizes complement activation. Materials of construction of these features and other features of the present disclosure may be high-density polyethylene, low-density polyethylene, ultrahigh molecular weight polyethylene, polypropylene, polypropylene copolymers, cyclo olefin copolymer, polyvinyl chloride plasticized, polyvinyl chloride unplasticized, polystyrene, acrylonitrile butadiene styrene copolymer, styrene-acrylonitrile copolymer, acrylonitrile styrene acrylate, methacrylate acrylonitrile butadiene styrene copolymer, styrene-butadiene copolymer, acrylics, polycarbonates, high heat polycarbonates, polyurethanes, acetals, nylon, poly butylene terephthalate, poly ethylene terephthalate, copolyesters, polysufones, polysulfone blends, polyphenylene sulfide, liquid crystalline polymer, polyetherimide, polyamide-imide, polyetheretherketone, polytetrafluoroethylene, fluorinated ethylene propylene, perfluoro alkoxy, ethylene chlorotrifluorothylene, ethylene tetrafluoroethylene, polyvinyl fluoride, polyvinylidene difluoride, polyvinylidene fluoride, silicones, urethane thermoplastic elastomer, copolyester thermoplastic elastomer, polyamide thermoplastic elastomer, syrenic thermoplastic elastomer, olefinic thermoplastic elastomer, PLLA, polylactic acid, polyhydroxybutyrate, polyglycolic acid, poly(lactic-co-glycolic acid), polycaprolactone, cellulose, cellulose diacetate, cellulose triacetate, polyethersulfone, polyethersulfone plus polyarylate (PEPA), polymethylmethacrylate, ethylene vinyl alcohol copolymers, polyacrylonitrile, polyester polymer alloy, as well as other suitable materials.

Each of the embodiments of FIGS. 1-17 may include alternative form factors, locations, alignments of the beads, bead packing arrangements, filter configurations, and/or membrane configurations. The porous filter and/or porous membranes 118 illustrated in the embodiments of FIGS. 12 and 16 may also include a sponge-like porous filter coated with complement receptors such as CRIg.

In some embodiments, a dialyzer is for use in peritoneal dialysis such as continuous ambulatory peritoneal dialysis (CAPD), continuous cycling peritoneal dialysis (CCPD), and/or intermittent peritoneal dialysis (IPD). For example, in some embodiments, peritoneal dialysis uses a dialysate comprising a complement receptor (e.g., CRIg). In certain embodiments, a complement receptor is associated with a polymeric substrate.

For example, FIG. 18 illustrates a peritoneal dialysis system 150 and associated method that can be used for removal of certain microbial content from the blood of the subject, rather than (or supplementary to) general purification as in traditional peritoneal dialysis. An associated method includes introducing a dialysate to a peritoneal cavity of the subject, wherein the dialysate comprises at least one complement receptor, such that the dialysate is in contact with the biological fluid; and removing the dialysate from the biological fluid after sufficient time to opsonize (or otherwise capture and/or deactivate) a pathogen. FIG. 18 shows an IV bag containing dialysate, introduced to the subject via a catheter 202 (e.g., by opening the line via the clamp 203). The dialysate enters the abdominal cavity 204. The blood of the subject stays in the arteries and veins that line the peritoneal cavity; the lining is the peritoneum 206. The pathogen leaches out of the blood into the dialysate where it is captured and/or deactivated. After sufficient time, the dialysate is drained from the peritoneal cavity, e.g., into a drain bag 207, thereby completing the “exchange”. The system may also employ continuous cyclic peritoneal dialysis (CCPD), also called automated peritoneal dialysis (APD), in which a machine 208 (e.g., with pumps, controllers, etc.) automatically performs the fluid exchanges. In another alternative, the system may employ continuous ambulatory peritoneal dialysis (CAPD) in which gravity moves fluid through the catheter and out of the abdomen.

### Methods

The present disclosure provides methods for treating a biological fluid of a subject suffering from or at risk for a microbial infection (e.g., a microbial infection in the blood).

In some embodiments, a subject for treatment with a method and/or system of the present disclosure has a microbial infection. In some embodiments, a subject has sepsis. In some embodiments, a subject for treatment with methods and systems of the present disclosure is at risk for a microbial infection, for example, a subject has been exposed to an antibiotic resistant infection, a subject is undergoing long-term dialysis, and/or a subject has recently been released from a hospital.

In some embodiments, a method of treating a subject with one or more clinically important bacilli, bacteria, viruses, and/or fungi, which may include: Acetobacter aurantius, Acinetobacter baumannii, Actinomyces israelii, Agrobacterium radiobacter, Agrobacterium tumefaciens, Anaplasma, Anaplasma phagocytophilum, Azorhizobium caulinodans, Azotobacter vinelandii, viridans streptococci, Bacillus, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, “Bacillus Thuringiensis”, Bacteroides, Bacteroides fragilis, Bacteroides gingivalis, Bacteroides melaninogenicus (now known as Prevotella melaninogenica), Bartonella, Bartonella henselae, Bartonella quintana, Bordetella, Bordetella bronchiseptica, Bordetella pertussis, Borrelia burgdorferi, Brucella, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila, Chlamydophila pneumoniae (previously called Chlamydia pneumoniae), Chlamydophila psittaci (previously called Chlamydia psittaci), Clostridium, Clostridium botulinum, Clostridium difficile, Clostridium perfringens (previously called Clostridium welchii), Clostridium tetani, Coronavirus (e.g., SARS-COV-2, previously called 2019-nCoV), Corynebacterium, Corynebacterium diphtheriae, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus gallinarum, Enterococcus maloratus, Escherichia coli, Francisella tularensis, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumoniae, Mycoplasma mexican, Neisseria, Neisseria gonorrhoeae, Neisseria meningitidis, Pasteurella, Pasteurella multocida, Pasteurella tularensis, Peptostreptococcus, Porphyromonas gingivalis, Prevotella melaninogenica (previously called Bacteroides melaninogenicus), Pseudomonas aeruginosa, Rhizobium radiobacter, Rickettsia, Rickettsia prowazekii, Rickettsia psittaci, Rickettsia quintana, Rickettsia rickettsii, Rickettsia trachomas, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Spirillum volutans, Staphylococcus, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Treponema pallidum, Treponema denticola, Thiobacillus, Vibrio, Vibrio cholerae, Vibrio comma, Vibrio parahaemolyticus, Vibrio vulnificus, Wolbachia, Yersinia, Yersinia enterocolitica, Yersinia pestis, and Yersinia pseudotuberculosis.

In some embodiments, a method of treating a subject with one or more clinically important viruses, which may include: Adenovirus, Herpes simplex, type 1, Herpes simplex, type 2, a coronavirus (e.g., SARS-CoV-2, previously called 2019-nCoV, Severe acute respiratory syndrome coronavirus (SARS-CoV), and Middle East Respiratory Syndrome Coronavirus (MERS-CoV)), Varicella-zoster virus, Epstein-Barr virus, Human cytomegalovirus, Human herpesvirus, type 8, Human papillomavirus, BK virus, JC virus, Smallpox, Hepatitis B virus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, and Banna virus.

In some embodiments, a method of treating a subject with one or more clinically important fungi, which may include: Candida, Candida albicans, Aspergillus, Aspergillus fumigatus, Aspergillus flavus, Aspergillus clavatus, Cryptococcus, Cryptococcus neoformans, Cryptococcus laurentii, Cryptococcus albidus, Cryptococcus gattii, Histoplasma, Histoplasma capsulatum, Pneumocystis, Pneumocystis jirovecii, Pneumocystis carinii, Stachybotrys, and Stachybotrys chartarum.

In some embodiments, a subject for treatment with a method and/or system of the present disclosure has a more than one infection. For example, it has been found that life-threatening secondary infections (e.g., MRSA, etc.) can occur during the coronavirus incubation period or during/after the course of COVID-19, the illness caused by SARS-CoV-2 (also called 2019-nCoV). The invention may be useful in the treatment of such primary and/or secondary infections. In some embodiments, a subject may have a primary viral infection (e.g., a SARS-CoV-2 infection) and a secondary bacterial infection (e.g., a drug-resistant microbial infection).

In some embodiments, a method for treating a biological fluid of a subject having a microbial disease comprises: contacting the biological fluid of the subject with a high surface area polymer having a substance immobilized thereupon or associated therewith, said substance comprising one or more complement receptors, thereby treating the microbial disease.

In some embodiments, the contacting step treats the subject of the infection (e.g., reducing the severity of the infection or eliminating the infection). In some embodiments, the contacting step isolates and/or removes invasive microbial content from the biological fluid. In some embodiments, the contacting step is performed under such conditions and for a time that all or substantial all of the invasive microbes are removed from the sample, thereby treating the microbial infection.

In certain embodiments, the contacting step comprises conducting the biological fluid of the subject through a filter (e.g., a membrane or other structure) comprising the high surface area polymer. In certain embodiments, the contacting step is conducted ex vivo. In certain embodiments, the contacting step is conducted in vivo.

In certain embodiments, the method further comprises returning the biological fluid to the subject following the contacting step.

In certain embodiments, the composition further comprises a drug or other agent that enhances the ability of innate immune cells in the biological fluid (e.g., neutrophils) and complement to kill bacteria in the biological fluid. In certain embodiments, the composition further comprises thymic stromal lymphopoietin (TSLP) (e.g., wherein the TSLP enhances the ability of innate immune cells in the biological fluid (e.g., neutrophils) and the complement receptor to kill bacteria in the biological fluid).

In certain embodiments, the treatment filter comprises a membrane comprising the high surface area polymer.

In certain embodiments, the contacting step comprises flowing a biological fluid through a treatment cartridge, where the cartridge comprises particles associated (e.g., coated) with one or more complement receptors. In some embodiments, the contracting step results in the binding of invasive microbial content (e.g., MRSA) from the biological fluid to the cartridge, thereby removing the invasive microbes from the biological fluid (e.g. blood).

In some embodiments, provided are methods of removing microbial content from a biological fluid from a subject. In some embodiments, a subject has a blood infection, and the method comprises removing the infectious microbe from the blood of the subject. In some embodiments, a subject has sepsis.

In some embodiments, methods of removing microbial content from a subject employ a hemodialysis or hemodialysis-like system, where blood is pumped from the body, filtered outside the body to remove certain microbial content, then returned to the body. For example, the filter and/or dialysate are formulated for removal of such microbial content as discussed in detail herein, rather than (or supplementary to) general purification as in traditional hemodialysis for subjects with kidney disease, damage, or failure.

In some embodiments, methods of removing microbial content from a subject employ a peritoneal dialysis or peritoneal dialysis-like system and/or method including: introducing a dialysate to a peritoneal cavity of the subject, wherein the dialysate comprises at least one complement receptor, such that the dialysate is in contact with the biological fluid; and removing the dialysate from the biological fluid after sufficient time to opsonize a pathogen. In traditional peritoneal dialysis, the dialysate is formulated to generally absorb waste and fluid from the blood using the peritoneum as a filter. However, in these embodiments, the dialysate is formulated for removal of certain microbial content from the blood of the subject, as discussed in detail herein, rather than (or supplementary to) general purification as in traditional peritoneal dialysis for subjects with kidney disease, damage, or failure. In some embodiments, peritoneal dialysis is or comprises continuous ambulatory peritoneal dialysis (CAPD), continuous cycling peritoneal dialysis (CCPD), and/or intermittent peritoneal dialysis (IPD).

Dialysis patients are at increased risk for blood infections, including MRSA. Dialysis poses an increased risk for infections as a result of impure dialysate and transient bacteremia caused by vascular access. Dialysis catheters disrupt the normal skin barrier and form a gateway for bacterial entry into the bloodstream. In some embodiments, provided methods are useful for treating dialysis patients at risk for a microbial infection, or otherwise at risk patients exposed to a microbial infection.

In some embodiments, a method includes treating the biological fluid (e.g., running through a system of the present disclosure and/or contacting the fluid with a filter or cartridge for a sufficient time) until a biological fluid is substantially free of the invasive microbial content. In some embodiments, substantially free of microbial content is >80% of the invasive microbial content removed, >85% of invasive microbial content removed, >90% of invasive microbial content removed, >95% of invasive microbial content removed, >96% of invasive microbial content removed, >97% of invasive microbial content removed, >98% of invasive microbial content removed, or >99% of invasive microbial content removed. In some embodiments, a polymer cartridge can be replaced until a biological fluid being treated by a system of the present disclosure (e.g., a dialysis system) is essentially completely free of microbial content.

In some embodiments, a subject has received or is to receive surgery, for example to remove microbial infection (e.g., MRSA) from solid tissues, such that the subject is treated by both a method of the present disclosure and surgery.

In some embodiments, a subject additionally has been treated with or is to be treated with antibiotics, such that the subject is treated with both a method of the present disclosure and antibiotics, said antibiotics including tetracyclines, aminoglycosides, macrolides, clindamycin, linezolid (oxazolidinono, chloramphenicol, streptogramins, quinilones, sulfonamides, trimethoprim, rifampin, vancomycin, trimethoprim/sulfamethoxazole, doxycycline, ceftobiprole, ceftaroline, clindamycin, dalbavancin, Daptomycin, fusidic acid, mupirocin, omadacycline, oritavancin, tedizolid, telavancin, and/or tigecycline.

In another aspect, the invention is directed to a method of operating the system of any of the embodiments described herein, the method comprising conducting a flow of the biological fluid over the surface of the high surface area polymer in the treatment filter to concentrate the one or more complement receptors onto the polymer.

In another aspect, the invention is directed to a method of operating the system of any of the embodiments described herein, the method comprising conducting a flow of the biological fluid over the surface of the high surface area polymer in the treatment filter, whereupon the polymer activates or enhances activation of the one or more complement components, thereby enhancing the killing of bacteria in the biological fluid by innate immune cells (e.g., neutrophils) in the biological fluid.

### Complement Components

The complement system is a part of the innate immune system that enhances (complements) the ability of antibodies and phagocytic cells to clear microbes and damaged cells from an organism, promotes inflammation, and attacks the pathogen's cell membrane. Complement proteins are present in blood and body fluids as inactive precursors but are rapidly activated upon contact with bacterial cells. The complement system is activated by microbial surfaces, which leads to a cascade of proteolytic reactions that coat the microbe. Thus, complement proteins can physically associate specifically with microbes, but not a subject's own cells.

In vivo complement activation enhances the immune response that takes place in the biological fluid to fight an infection. For example, activated complement may improve various host defenses against an infection, such as opsonization; chemotaxis and activation of leukocytes (e.g., neutrophils); and/or lysis of bacteria, thereby enhancing a subject's immune response to a microbial infection.

In some embodiments, one or more complement receptors are isolated and/or identified from a subject to be treated with a method or system of the present disclosure. In some embodiments, one or more complement receptors are isolated and/or identified from a different subject than the one to be treated with a method or system of the present disclosure. In some embodiments, one or more complement receptors are recombinantly produced. In some embodiments, one or more complement receptors are engineered.

In certain embodiments, the method comprises immobilizing the substance comprising the one or more complement receptors on the high surface area polymer in a first step (e.g., loading phase), then performing the contacting step, subsequent to the first step, to treat the biological fluid (e.g., treatment phase). The loading phase may be performed using blood or other biological fluid from the same subject whose biological fluid is treated during the treatment phase, or the loading phase may be performed using blood or other biological fluid from a different subject than the subject whose biological fluid is treated during the treatment phase. In some embodiments, the complement receptors are otherwise isolated and/or engineered and are loaded onto the polymer.

Complement receptors, as used herein, include any receptors capable of binding one or more proteins of the complement system. Generally, complement receptors are membrane proteins expressed on the surface of immune cells. In vivo, complement receptors bind proteins of the complement system, and can thus detect pathogens without mediation by antibodies. Complement receptors interact specifically with complement factors, which in vivo may lead to removal of antigen from the circulation. Complement receptors are listed in Table 1 below.

TABLE 1
Example complement receptors

| | | CD | | |
|---|---|---|---|---|
| Receptor | Ligand | number | Protein superfamily | Function |
| CR1 | C3b | CD35 | Regulators of | Promotion of phagocytosis, |
| | | | complement activation | immune complex (IC) clearance, |
| | | | | processing of IC-bound C3b |
| CR2 | C3dg | CD21 | Regulators of | B-cell proliferation, alternative |
| | (iC3b) | | complement activation | pathway activation |
| CR3 | iC3b | CD11b/ | β2 integrin | reactive oxygen metabolites/nitric |
| | | CD18 | | oxide (ROM/NO) synthesis, |
| | | | | degranulation |
| CR4 | iC3b | CD11c/ | β2 integrin | Phagocytosis, leucocyte |
| | | CD18 | | migration |
| C3aR | C3a | — | G protein-coupled | NO synthesis |
| | | | receptors | |
| C5aR | C5a; | CD88 | G protein-coupled | Leucocyte chemoattraction, |
| | C5a-desarg | | receptors | degranulation |
| cC1qR | C1q, MBL, | — | — | Chemotaxis, promotion of |
| | SPA | | | phagocytosis, ROM |
| C1qRp | C1q, MBL, | CD93 | — | Synthesis, platelet aggregation, |
| | SPA | | | leucocyte migration |

In some embodiments, one or more complements receptor is selected from: CR1, CR2, CR3, CR4, CR3aR, CR5aR, CRIg, C1qR, and C1qRp. In some certain embodiments, a complement receptor is a CR1 and/or a CR2.

In some embodiments, a complement receptor is a Complement Receptor of the Immonoglobulin Superfamily (CRIg) protein or a binding portion thereof. CRIg is expressed on Kupffer cells (tissue-resident macrophages of the liver) in vivo, and consists of a two Ig-like repeats and a C-terminal extension that tethers it to the cell plasma membrane. The active part of CRIg comprises a N-terminal Ig-like domain, which is ˜15 kDa. This region of CRIg can be expressed in E. coli, and modified accordingly to link it to the surface of the membrane via cysteine-based chemistries. CRIg binds to the C3c region of the molecule via a site that is hidden in native C3, but exposed in C3b and iC3b. In some embodiments, a complement receptor comprising a N-terminal Ig-like domain of CRIg. In some embodiments, a CRIg or binding portion thereof binds to C3b with a Kd of about 10 nM, or less.

In embodiments, a complement receptor is a Staphylococcal Complement Inhibitor (SCIN). SCIN is expressed by S. aureus, and is ˜10 kDa. SCIN also can be expressed in E. coli and modified accordingly to link it to the surface of the membrane via cysteine-based chemistries. SCIN binds selectively to C3b and iC3b, but does not bind C3 or C3d. SCIN can also induce dimerization of C3b. In some embodiments, a SCIN or binding portion thereof is binds to C3b with a Kd of about 100 nM, or less.

In some embodiments, a complement receptor is recombinant (e.g., made by recombinant techniques). In some embodiments, a complement receptor is isolated and/or identified from a subject to be treated by the method. In some embodiments, a complement receptor is isolated and/or identified from a subject other than the one to be treated by the method.

In some embodiments, particles (e.g., polymer particles and/or magnetic particles) and/or fibers (e.g., polymer fibers, e.g., hollow polymer fibers) are associated with (e.g., coated with) one or more complement receptors. In some embodiments, additional complement protein(s) are introduced into a biological fluid concurrently with or prior to contacting the biological fluid with the complement-receptor associated particles (e.g., flowing through a cartridge). In some embodiments, one or more of a C1 protein, C3 protein, and C4 protein are introduced into a biological fluid prior to or simultaneous with a contacting step.

### High Surface Area Polymers

In some embodiments, a high surface area polymer is a biocompatible polymer. In certain embodiments, the polymer comprises poly (methyl methacrylate) (PMMA). In certain embodiments, the polymer comprises nylon (e.g., Nylon-6, 6). In certain embodiments, the polymer comprises one or more members selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof.

In some embodiments, high surface area polymer comprises particles (e.g., microspheres). In some embodiments, high surface area polymer particles are made, for example, by dissolving polymer in a solvent, injecting the polymer into water via turbulent jet, then drying the precipitate. For example, high surface area nylon 6,6 can be made by dissolving the nylon 6,6 polymer in formic acid, injecting the dissolved polymer into water via turbulent jet, then drying the precipitate. High surface area PMMA can be made, for example, by dissolving PMMA in organic solvent (e.g., tetrahydrofuran, THF), injecting the dissolved PMMA into water via turbulent jet, then drying the precipitate. Other biocompatible polymers may also be used, in certain embodiments, for example, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), or a mixture and/or copolymer thereof.

In some embodiments, a high surface area polymer meet requirements for biocompatibility as set forth in designations set forth by the General Chapter of the United States Pharmacopeia (USP). In some embodiments, a high surface area polymer meets USP designation requirements of at least a USP Class IV material. In some embodiments, a high surface area polymer meets USP designation requirements of a USP Class IV, USP Class V, or USP Class VI material. In certain embodiments, a high surface area polymer meets USP designation requirements of a USP Class IV material.

In some embodiments, high surface area polymer comprises porous fibers, e.g., hollow fibers, e.g., wherein a cartridge of the system comprises hollow fiber membranes. The polymeric fibers may have pores that allow non-cellular biological fluid and microbes therein to pass through, but which do not allow cells or other larger structures in the biological fluid medium to pass through.

In some embodiments, a high surface area polymer has surface area within a range of about 50,000 cm²/g to about 1,000,000 cm²/g. In some embodiments, a high surface area polymer has surface area within a range bounded by a lower limit and an upper limit, the upper limit being larger than the lower limit. In some embodiments, the lower limit may be about 50,000 cm²/g, about 100,000 cm²/g, about 150,000 cm²/g, about 200,000 cm²/g, about 250,000 cm²/g, about 300,000 cm²/g, about 400,000 cm²/g, or about 500,000 cm²/g. In some embodiments, the upper limit may be about 150,000 cm²/g, about 200,000 cm²/g, about 250,000 cm²/g, about 300,000 cm²/g, about 400,000 cm²/g, or about 500,000 cm²/g, about 600,000 cm²/g, about 700,000 cm²/g, about 800,000 cm²/g, about 900,000 cm²/g, or about 1,000,000 cm²/g.

In certain embodiments, the high surface area polymer has surface area of at least 50,000 cm²/g (e.g., more preferably, at least 100,000 cm²/g, more preferably, at least 150,000 cm²/g, or more preferably, at least 200,000 cm²/g).

In some embodiments, a high surface area polymer is in particle form (e.g., microspheres). In some embodiments, high surface area polymer particles have an average dimension (e.g., diameter) within a range of about 0.01 μm to about 5 μm. In some embodiments, high surface area polymer particles have an average dimension (e.g., diameter) within a range bounded by a lower limit and an upper limit, the upper limit being larger than the lower limit. In some embodiments, the lower limit may be about 0.01 μm, about 0.025 μm, about 0.05 μm, about 0.075 μm, about 0.1 μm, about 0.2 μm, about 0.3 μm, about 0.4 μm, about 0.5 μm, about 0.6 μm, about 0.7 μm, about 0.8 μm, about 0.9 μm, or about 1 μm. In some embodiments, the upper limit may be about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, or about 5 μm.

In certain embodiments, the high surface area polymer comprises particles having an average dimension (e.g., diameter) of less than 1 micrometer.

In some embodiments, high surface area polymer particles further comprise magnetic material. In some embodiments, particles useful in methods and systems of the present disclosure comprise magnetic beads coated with polymer.

In some embodiments, methods and systems of the present disclosure include a high surface area polymer with one or more complement receptors immobilized on, or otherwise associated with, the polymer.

In some embodiments, a high surface area polymer is contacted with one or more complement receptors such that the one or more complement receptors covalently associate with the polymer (e.g., with the surface of the polymer particles). In some embodiments, a high surface area polymer is contacted with one or more complement receptors such that the one or more complement receptors non-covalently associate with the polymer (e.g., with the surface of the polymer particles).

In certain embodiments, the polymer activates or enhances activation of the one or more complement receptors, thereby enhancing the killing of bacteria in the biological fluid by innate immune cells (e.g., neutrophils) in the biological fluid.

In certain embodiments, the polymer has a coating (e.g., at least a partial coating) on its surface. In some embodiments a coating is or comprises sialic acid, fH protein (or a fragment thereof), a mini fH protein, or a fH-recruiting peptide.

In certain embodiments, the polymer has a coating (e.g., at least a partial coating) on its surface that selectively binds the one or more complement receptors from the biological fluid onto the surface of the polymer (e.g., wherein the method comprises conducting a flow of the biological fluid over the surface of the polymer to concentrate the one or more complement receptors onto the polymer, whereupon the polymer activates or enhances activation of the one or more complement receptors, thereby enhancing the killing of bacteria in the biological fluid by innate immune cells (e.g., neutrophils) in the biological fluid).

### Magnetic Particles

In some embodiments, methods and systems of the present disclosure include magnetic beads with one or more complement receptors immobilized on, or otherwise associated with, the magnetic beads.

In some embodiments, magnetic beads associated with one or more complement receptors are generated from commercially available beads (e.g., ThermoFisher Dynabeads). In some embodiments, magnetic beads are contacted with one or more complement receptors such that the one or more complement receptors covalently associate with the magnetic beads. In some embodiments, magnetic beads are contacted with one or more complement receptors such that the one or more complement receptors non-covalently associate with the magnetic beads.

In some embodiments, magnetic beads have an average dimension (e.g., diameter) within a range of about 0.01 μm to about 10 μm. In some embodiments, magnetic beads have an average dimension (e.g., diameter) within a range bounded by a lower limit and an upper limit, the upper limit being larger than the lower limit. In some embodiments, the lower limit may be about 0.01 μm, about 0.025 μm, about 0.05 μm, about 0.075 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, or about 2 μm. In some embodiments, the upper limit may be about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about 8 μm, about 9 μm, or about 10 μm.

In certain embodiments, the magnetic beads have a coating (e.g., at least a partial coating) on their surface that selectively binds the one or more complement receptors.

### Microbial Infections

Methods and systems of the present disclosure are useful for treating or removing microbes in a biological fluid. For example, in some embodiments, methods and systems may activate complement to employ the innate immune system to target the microbe for destruction. In some embodiments, methods and systems may isolate and/or remove microbes from biological fluid (e.g., by filtering or trapping microbes in a cartridge).

In some embodiments, a microbe in a biological fluid of a subject is a gram negative microbe. In some embodiments, a microbe in a biological fluid of a subject is a gram positive microbe, such as, for example Staphyloccocus, Streptococcus, and Enterococcus species.

In some embodiments, methods and systems are useful for treating or removing microbes in the blood of a subject, for example, a subject with sepsis.

In some embodiments, a biological fluid of a subject comprises one or microbes, such as Escherichia coli (E. coli), Group B Streptococcus, Staphylococcus aureus, Group A Streptococcus, Group B Streptococcus, Pseudomonas aeruginosa, Klebsiella pneumoniae, Proteus mirabilis, and/or Salmonella

In certain embodiments, the subject has an antibiotic-resistant infection (e.g., Methicillin-Resistant Staphylococcus aureus, Streptococcus pneumoniae, Carbapenem-Resistant Enterobacteriaceae, Clostridium difficile, Drug-Resistant Neisseria gonorrhoeae, Drug-Resistant Malaria, Multi-drug resistant (MDR) or extensively drug resistant (XDR) Tuberculosis).

In certain embodiments, the biological fluid comprises methicillin-resistant Staphylococcus aureus (MRSA), and wherein the system and/or method enhances the killing of MRSA in the biological fluid.

In certain embodiments, the biological fluid comprises one or more infectious microbes selected from the group consisting of Methicillin-Resistant Staphylococcus aureus, Streptococcus pneumoniae, Carbapenem-Resistant Enterobacteriaceae, Clostridium difficile, Drug-Resistant Neisseria gonorrhoeae, Drug-Resistant Malaria, and Multi-drug resistant (MDR) or extensively drug resistant (XDR) Tuberculosis, and wherein the system enhances the killing of the one or more infectious microbes.

In some embodiments, an infectious microbe is a yeast. In some embodiments, a subject has candidemia, i.e., presence of one or more Candida species in the blood. In some embodiments, a biological fluid comprises an infectious Candida species. In some embodiments, an infectious microbe is or comprises an infectious fungus selected from C. albicans, C. glabrata, C. parapsilosis, C. tropicalis, C. auris, and C. krusei. In some embodiments, an infectious fungus is resistant to anti-fungal treatment, such as an azole (e.g., fluconazole, itraconazole, isavuconazole, posaconazole, and voriconazole), a polyene (e.g., amphotericin B and its lipid formulations), an echinocandin (e.g., anidulafungin, caspofungin, and micafungin), a pyrimidine analogue, (e.g., flucytosine). In certain embodiments, an infectious microbe is a fluconazole-resistant Candida. In some embodiments, an infectious fungus is multi-drug resistant, in that it is resistant to at least 1 drug agent in at least two drug classes selected from azoles, polyenes, echinocandins, and pyrimidine analogues.

In some embodiments, a microbe in a biological fluid of a subject is a virus. In some embodiments, a subject has a viral infection, such as an infection with Adenovirus, Herpes simplex, type 1, Herpes simplex, type 2, a coronavirus (e.g., severe acute respiratory syndrome coronavirus 2 (SARS-CoV-2, previously called 2019-nCoV), Severe acute respiratory syndrome coronavirus (SARS-CoV), and Middle East Respiratory Syndrome Coronavirus (MERS-CoV)), Varicella-zoster virus, Epstein-Barr virus, Human cytomegalovirus, Human herpesvirus, type 8, Human papillomavirus, BK virus, JC virus, Smallpox, Hepatitis B virus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, and Banna virus.

### Flow Rate Simulations

Flow rates were simulated to compute how long it would take to remove all the MRSA from a subject using the methods, systems and apparatus discussed herein (see Example 1 below). The simulations employed a simplified mathematical model of Monod kinetics, and predicted that greater blood flow rates greatly reduced the time required. The simulation used the following inputs: a growth rate of 0.13 h⁻¹, a blood flow rate of 300 ml/min, an 85% efficacy per pass, 5000 ml of blood volume, and 500,000 CFU initially in the blood stream. The simulation predicted that the time required to clear out virtually all the pathogens went down from 4.75 hours to 1.25 hours as the flow rate was increased from 300 ml/min to 1200 ml/min.

### Example 1

In silico experiments were performed using a simplified mathematical model of Monod kinetics which allows for pathogen capture with simultaneous growth of the pathogen. The model can be described by

$$\frac{dCp}{dt} = {C}_{p} ( μ - \frac{F φ}{V} )$$

where Cₚ is the concentration of the pathogen, is the growth rate of the pathogen (0.13 h⁻¹ for S. aureus), F is the flow rate in ml/hr, φ is the pathogen removal efficiency in a single round pass and Vis the blood volume. The model does not assume that the patient continually seeds the blood with additional pathogens from another focal infection point. Furthermore the model does not assume that the body removes pathogens or that antibiotics are applied simultaneously.

Assuming a growth rate of 0.13 h⁻¹, flow rate of 300 ml/min, an 85% efficacy per pass, 5000 ml of blood volume, and 500,000 CFU initially in the blood stream, the simulation predicts that the patient can be treated for 4.75 hours and have less than 1 CFU left in the blood assuming the patient's body does not remove any pathogen. The treatment time will be less if the patient's body simultaneously removes pathogens as well.

| | Number of Bacteria | Percent of original |
|---|---|---|
| Treatment Time (hours) | (CFU) | Bacteria |
| 0 | 500000 | 100.000% |
| 0.25 | 240353 | 48.071% |
| 0.5 | 115539 | 23.108% |
| 0.75 | 55540 | 11.108% |
| 1 | 26699 | 5.340% |
| 1.25 | 12834 | 2.567% |
| 1.5 | 6169 | 1.234% |
| 1.75 | 2966 | 0.593% |
| 2 | 1426 | 0.285% |
| 2.25 | 685 | 0.137% |
| 2.5 | 329 | 0.066% |
| 2.75 | 158 | 0.032% |
| 3 | 76 | 0.015% |
| 3.25 | 37 | 0.007% |
| 3.5 | 18 | 0.004% |
| 3.75 | 8 | 0.002% |
| 4 | 4 | 0.001% |
| 4.25 | 2 | 0.000% |
| 4.5 | 1 | 0.000% |
| 4.75 | 0 | 0.000% |

### Example 2

Assuming the same variables as Example 1, except that the flow rate is increased to 1200 ml/min, less than 1 CFU of S. aureus would be left in the patient after 1.25 hours.

| Treatment Time | Number of Bacteria | Percent of original |
|---|---|---|
| (hours) | (CFU) | Bacteria |
| 0 | 500000 | 100.000% |
| 0.25 | 24218 | 4.844% |
| 0.5 | 1173 | 0.235% |
| 0.75 | 57 | 0.011% |
| 1 | 3 | 0.001% |
| 1.25 | 0 | 0.000% |

### Example 3

Assuming the same variables as Example 1, except that the flow rate is increased to 1200 ml/min for the first 15 min and then the remaining time it is 300 ml/min, less than 1 CFU of S. aureus would be left in the patient after 4 hours.

| | Number of Bacteria | Percent of original |
|---|---|---|
| Treatment Time (hours) | (CFU) | Bacteria |
| 0 | 500000 | 100.000% |
| 0.25 | 24218 | 4.844% |
| 0.5 | 11642 | 2.328% |
| 0.75 | 5596 | 1.119% |
| 1 | 2690 | 0.538% |
| 1.25 | 1293 | 0.259% |
| 1.5 | 622 | 0.124% |
| 1.75 | 299 | 0.060% |
| 2 | 144 | 0.029% |
| 2.25 | 69 | 0.014% |
| 2.5 | 33 | 0.007% |
| 2.75 | 16 | 0.003% |
| 3 | 8 | 0.002% |
| 3.25 | 4 | 0.001% |
| 3.5 | 2 | 0.000% |
| 3.75 | 1 | 0.000% |

### Example 4

Assuming the same variables as Example 1, except the flow rate is increased to 1200 ml/min after 1 hour, less than 1 CFU of bacteria would be left in the patient after 2 hours.

| Treatment Time | Number of Bacteria | Percent of original |
|---|---|---|
| (hours) | (CFU) | Bacteria |
| 0 | 500000 | 100.000% |
| 0.25 | 240353 | 48.071% |
| 0.5 | 115539 | 23.108% |
| 0.75 | 55540 | 11.108% |
| 1 | 26699 | 5.340% |
| 1.25 | 1293 | 0.259% |
| 1.5 | 63 | 0.013% |
| 1.75 | 3 | 0.001% |
| 2 | 0 | 0.000% |

### Example 5

Assuming the same variables as Example 1, except the efficacy per pass is increased to 95% after 1 hour, less than 1 CFU of S. aureus would be left in the blood after 4.5 hours.

| Treatment Time | Number of Bacteria | Percent of original |
|---|---|---|
| (hours) | (CFU) | Bacteria |
| 0 | 500000 | 100.000% |
| 0.25 | 240353 | 48.071% |
| 0.5 | 115539 | 23.108% |
| 0.75 | 55540 | 11.108% |
| 1 | 26699 | 5.340% |
| 1.25 | 11730 | 2.346% |
| 1.5 | 5153 | 1.031% |
| 1.75 | 2264 | 0.453% |
| 2 | 995 | 0.199% |
| 2.25 | 437 | 0.087% |
| 2.5 | 192 | 0.038% |
| 2.75 | 84 | 0.017% |
| 3 | 37 | 0.007% |
| 3.25 | 16 | 0.003% |
| 3.5 | 7 | 0.001% |
| 3.75 | 3 | 0.001% |
| 4 | 1 | 0.000% |
| 4.25 | 1 | 0.000% |
| 4.5 | 0 | 0.000% |

The blood flow rate may be steady or variable over the course of the treatment. Flow rates may be optimized to remove pathogens as quickly as possible, which may not necessarily result in higher flow rates equating to higher removal rates (since blood residence time within the filter cartridge 34 (and/or hollow fiber membranes 100) also has an effect on removal rate). Flow rates around 300 ml/min can be obtained peripherally at the femoral or jugular access points, while flow rates around 1200 ml/min can be obtained via a central line catheter to the heart. According to the present embodiments, access (i.e., where blood is extracted from the subject) may be femoral, radial, jugular, and/or via a central line. Ideally patients with access points for other treatments will be connected to the system 10 at an access point which is unoccupied. In some embodiments, multiple access points may be used simultaneously in order to increase the flow rate. In other embodiments, different gauge needles (for example 14 G or 17 G needles) may be used to increase or decrease the flow rate, depending on the circumstances. In some embodiments, the system may include blood and/or fluid heaters and coolers to maintain the temperature at about 37° C., or in some cases at temperatures anywhere from about 30° C. to about 45° C. In some embodiments, treatment times may be expected to be from about 2 hours to about 5 hours. However, in other embodiments, the treatment time could vary from anywhere from about 5 minutes to about 24 hours, depending on the severity of the infection, as well as other conditions that may vary from subject to subject.

### Certain Definitions

In order for the present disclosure to be more readily understood, certain terms are first defined below. Additional definitions for the following terms and other terms are set forth throughout the specification.

As used herein, the term “administration” refers to the administration of a composition to a subject or system. Those of ordinary skill in the art will be aware of a variety of routes that may, in appropriate circumstances, be utilized for administration to a subject, for example a human. For example, in certain embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In certain embodiments, administration may involve dosing that is intermittent (e.g., a plurality of doses separated in time) and/or periodic (e.g., individual doses separated by a common period of time) dosing. In certain embodiments, administration may involve continuous dosing (e.g., perfusion) for at least a selected period of time.

As used herein, the term “biocompatible” refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

As used herein, the term “biological fluid” refers to blood (e.g., unmodified whole blood, modified blood), serum, plasma, cerebrospinal fluid, lymph, synovial fluid, or amniotic fluid.

A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

As used herein, the term “designed” refers to an agent (i) whose structure is or was selected by the hand of man; (ii) that is produced by a process requiring the hand of man; and/or (iii) that is distinct from natural substances and other known agents.

As used herein, the term “low flow rate” refers to a non-zero (e.g., no less than 1 ml/min) flow rate less than about 100 ml/min, or less than about 50 ml/min, or less than about 40 ml/min, or less than about 30 ml/min, or less than about 20 ml/min, or less than about 15 ml/min, or less than about 10 ml/min.

As used herein, the term “patient” or “subject” (used interchangeably herein) refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In certain embodiments, a patient is a human. In certain embodiments, a patient is suffering from or susceptible to one or more disorders or conditions. In certain embodiments, a patient displays one or more symptoms of a disorder or condition. In certain embodiments, a patient has been diagnosed with one or more disorders or conditions. In certain embodiments, the disorder or condition is or includes bacterial infection. In certain embodiments, the patient is receiving or has received certain therapy to diagnose and/or to treat a disease, disorder, or condition.

As used herein, the term “substantially” refers to the qualitative condition of exhibiting total or near-total extent or degree of a characteristic or property of interest.

As used herein, the term “therapeutically effective amount” or “therapeutically effective concentration” refers to an amount or concentration that produces the desired effect for which it is administered or otherwise used. In certain embodiments, the term refers to an amount or concentration that is sufficient, when administered or otherwise used for a population suffering from or susceptible to a disease, disorder, and/or condition in accordance with a therapeutic dosing regimen or other treatment program, to treat the disease, disorder, and/or condition. In certain embodiments, a therapeutically effective amount or concentration is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered or otherwise used for patients in need of such treatment. In certain embodiments, reference to a therapeutically effective amount may be a reference to an amount or concentration as measured in a medium (e.g., filter) that comes into contact with the biological fluid (e.g., blood, serum, plasma) of the subject during treatment. In certain embodiments, reference to a therapeutically effective amount may be a reference to an amount or concentration as measured in one or more specific tissues (e.g., a tissue affected by the disease, disorder or condition) or fluids (e.g., blood (e.g., unmodified whole blood, modified blood), serum, plasma, cerebrospinal fluid, lymph, synovial fluid, or amniotic fluid). Those of ordinary skill in the art will appreciate that, in certain embodiments, a therapeutically effective amount of a particular agent or therapy may be formulated and/or administered in a single dose or otherwise used in a single treatment session. In certain embodiments, a therapeutically effective agent may be formulated and/or administered in a plurality of doses, for example, as part of a dosing regimen, or may be used in multiple treatment sessions.

As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In certain embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In certain embodiments, treatment may be of a subject who has been diagnosed as suffering from the relevant disease, disorder, and/or condition. In certain embodiments, treatment may be of a subject known to have one or more susceptibility factors that are statistically correlated with increased risk of development of the relevant disease, disorder, and/or condition. In certain embodiments, treatment is conducted in a single session. In certain embodiments, treatment is conducted in a series of sessions. In certain embodiments, treatment is conducted by contacting a biological fluid of the subject with a filter containing a high surface area polymer, e.g., polymer with complement protein (optionally, with one or more other agents) immobilized thereupon or otherwise associated therewith. One or more of the high surface area polymer, the complement protein, and the optional agent(s) may be present in the filter in a therapeutically effective amount and/or concentration.

### CONSTRUCTIVE EXAMPLES

The following examples are provided to illustrate, but not limit, the claimed invention.

### Example 1

The present example describes preparation of exemplary polymer particles for use system for treating a biological fluid in the context of the present disclosure. Provided herein are polymer particles with a complement receptor immobilized thereon. Specifically, this example describes production of particles with Complement Receptor of the Immunoglobulin superfamily (CRIg) covalently or non-covalently immobilized thereon. Optionally, polymer particles can be treated to prevent complement activation (e.g., with a coating is or comprises sialic acid, fH protein (or a fragment thereof), a mini fH protein, or a fH-recruiting peptide). For example, CRIg may be charged so that it non-covalently associates with the polymer particles. Alternatively, CRIg may be covalently attached to the polymer particles using a linker agent. Suitable linker agents are known in the art.

### Example 2

The present example describes in vitro confirmation that exemplary polymer particles of the present disclosure can bind to model opsonized particles (e.g., model opsonized microbial particles). Specifically, this example describes the ability of exemplary polymer particles with CRIg immobilized thereon to bind to model complement-opsonized particles (e.g., C3b-coated MRSA). A container containing exemplary polymer particles with CRIg immobilized thereon (e.g., as described in Example 1) will be contacted with a fluid (e.g., a biological fluid, e.g., blood and/or serum). Polymer particles can be treated to prevent complement activation (e.g., with a coating is or comprises sialic acid, fH protein (or a fragment thereof), a mini fH protein, or a fH-recruiting peptide). A test fluid containing model opsonized particles (e.g., a biological fluid, e.g., blood and/or serum) is flowed over the polymer particles at different rates (e.g., 500 ml/min and 1200 ml/min). Polymer particles with a complement control protein, Factor H, immobilized thereon can serve as a control.

### Example 3

The present example describes in vivo confirmation that immobilized CRIg (e.g., on a high surface area polymer particle) can bind to model C3b-opsonized particles. Specifically, this example describes treatment of non-human primates with a blood infection using an exemplary system of the present disclosure. Blood from infected non-human primates can be treated by a dialysis system of the present disclosure, which includes a cartridge containing CRIg coated polymeric particles.

### EQUIVALENTS

It is to be understood that while the disclosure has been described in conjunction with the detailed description thereof, the foregoing description is intended to illustrate and not limit the scope of the invention(s). Other aspects, advantages, and modifications are within the scope of the following claims.

What is claimed is:

1. A system for removing microbes from a biological fluid of a subject, the system comprising:
at least one filter cartridge comprising a plurality of beads with at least one complement receptor attached thereto or otherwise associated therewith; and
at least one tubing line for conducting the biological fluid from the subject to the at least one filter cartridge (e.g., and, in certain embodiments, back to the subject).

2. The system of claim 1, wherein the at least one complement receptor comprises at least one of CR1, CR2, CR3, CR4, CR3aR, CR5aR, C1qR, C1qRp, CRIg, and a complement binding portion of any thereof.

3. The system of claim 1 or 2, wherein at least one bead of the plurality of beads has attached thereto (or otherwise associated therewith) at least one complement inhibitor selected from the group consisting of sialic acid, Factor H protein (or a fragment thereof), a mini fH protein, and a fH-recruiting peptide.

4. The system of any one of the preceding claims, wherein the plurality of beads comprises a high surface area polymer.

5. A system for removing microbes from a biological fluid of a subject, the system comprising:
at least one filter cartridge having a chamber comprising at least one porous membrane with at least one complement receptor disposed thereon or therewithin, attached thereto, or otherwise associated therewith; and
at least one tubing line for conducting the biological fluid from the subject to the at least one filter cartridge (e.g., and, in certain embodiments, back to the subject).

6. The system of claim 5, wherein the at least one complement receptor comprises at least one of CR1, CR2, CR3, CR4, CR3aR, CR5aR, C1qR, C1qRp, CRIg, and a complement binding portion of any thereof.

7. The system of claim 5 or 6, wherein the at least one porous membrane has disposed thereon or therewithin at least one complement inhibitor selected from the group consisting of sialic acid, Factor H protein (or a fragment thereof), a mini fH protein, and a fH-recruiting peptide.

8. The system of any one of claims 5 to 7, wherein the at least one porous membrane comprises a high surface area polymer.

9. A bead for use in a system for removing microbes from a biological fluid of a subject, the bead having at least one complement receptor attached thereto or otherwise associated therewith.

10. The bead of claim 9, wherein the at least one complement receptor comprises at least one of CR1, CR2, CR3, CR4, CR3aR, CR5aR, C1qR, C1qRp, CRIg, and a complement binding portion of any thereof.

11. The bead of claim 9 or 10, wherein the bead comprises a diameter from about 1 micron to about 1000 microns.

12. The bead of any one of claims 9 to 11, wherein the bead has attached thereto or associated therewith at least one complement inhibitor selected from the group consisting of sialic acid, Factor H protein (or a fragment thereof), a mini fH protein, and a fH-recruiting peptide.

13. The bead of any one of claims 9 to 12, wherein the bead comprises a high surface area polymer.

14. A filter cartridge for use in a system for removing microbes from a biological fluid of a subject, the filter cartridge comprising at least one bead, the at least one bead comprising at least one complement receptor.

15. The filter cartridge of claim 14, wherein the at least one complement receptor comprises at least one of CR1, CR2, CR3, CR4, CR3aR, CR5aR, C1qR, C1qRp, CRIg, and a complement binding portion of any thereof.

16. The filter cartridge of claim 14 or 15, wherein the at least one bead has attached thereto or associated therewith at least one complement inhibitor selected from the group consisting of sialic acid, Factor H protein (or a fragment thereof), a mini fH protein, and a fH-recruiting peptide.

17. The filter cartridge of any one of claims 14 to 16, wherein the at least one bead comprises a high surface area polymer.

18. A method for treating a biological fluid of a subject having a microbial disease (e.g., extracorporeal treatment), the method comprising:
contacting the biological fluid of the subject with a high surface area polymer having a substance immobilized thereupon or associated therewith, said substance comprising one or more complement receptors.

19. The method of claim 18, further comprising administering at least one drug to the subject.

20. The method of claim 19, wherein the at least one drug comprising an antibiotic selected from the group consisting of: tetracyclines, aminoglycosides, macrolides, clindamycin, linezolid (oxazolidinone), chloramphenicol, streptogramins, quinilones, sulfonamides, trimethoprim, rifampin, vancomycin, trimethoprim/sulfamethoxazole, doxycycline, ceftobiprole, ceftaroline, clindamycin, dalbavancin, Daptomycin, fusidic acid, mupirocin, omadacycline, oritavancin, tedizolid, telavancin, and tigecycline.

21. The method of any one of claims 18 to 20, wherein the one or more complement receptors comprises at least one of CR1, CR2, CR3, CR4, CR3aR, CR5aR, C1qR, C1qRp, CRIg, and a complement binding portion of any thereof.

22. The method of any one of claims 18 to 21, wherein the high surface area polymer has attached thereto or associated therewith at least one complement inhibitor selected from the group consisting of sialic acid, Factor H protein (or a fragment thereof), a mini fH protein, and a fH-recruiting peptide.

23. The method of any one of claims 18 to 22, wherein the biological fluid is a member selected from the group consisting of blood, serum, plasma, cerebrospinal fluid, lymph, synovial fluid, and amniotic fluid.

24. The method of any one of claims 18 to 23, wherein the complement receptor is CRIg or a complement-binding portion thereof.

25. The method of any one of claims 18 to 24, wherein the substance immobilized on the high surface area polymer comprises CRIg and a complement inhibitor selected from: sialic acid, Factor H protein (or a fragment thereof), a mini fH protein, or a fH-recruiting peptide.

26. The method of any one of claims 18 to 25, wherein the high surface area polymer has surface area of at least 50,000 cm²/g (e.g., more preferably, at least 100,000 cm²/g, more preferably, at least 150,000 cm²/g, or more preferably, at least 200,000 cm²/g).

27. The method of any one of claims 18 to 26, wherein the high surface area polymer comprises particles having an average dimension (e.g., diameter) of less than 1 micrometer.

28. The method of any one of claims 18 to 27, wherein the polymer comprises one or more members selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof.

29. The method of any one of claims 18 to 28, wherein the polymer comprises nylon (e.g., Nylon-6, 6) and/or poly (methyl methacrylate) (PMMA).

30. The method of any one of claims 18 to 29, wherein the biological fluid comprises bacteria, virus, fungus, and/or protozoa, such as Methicillin-Resistant Staphylococcus aureus, Streptococcus pneumoniae, Carbapenem-Resistant Enterobacteriaceae, Clostridium difficile, Drug-Resistant Neisseria gonorrhoeae, Drug-Resistant Malaria, Tuberculosis (e.g., MDR or XDR tuberculosis), coronavirus (e.g., SARS-CoV-2), or an infectious Candida species.

31. The method of any one of claims 18 to 30, wherein the subject has an antibiotic-resistant infection (e.g., Methicillin-Resistant Staphylococcus aureus, Streptococcus pneumoniae, Carbapenem-Resistant Enterobacteriaceae, Clostridium difficile, Drug-Resistant Neisseria gonorrhoeae, Drug-Resistant Malaria, Multi-drug resistant (MDR) or extensively drug resistant (XDR) Tuberculosis), and wherein the contacting step treats the subject of the infection (e.g., reducing the severity of the infection or eliminating the infection).

32. The method of any one of claims 18 to 31, wherein the biological fluid comprises methicillin-resistant Staphylococcus aureus (MRSA), and wherein the contacting step enhances attachment of the at least one complement receptor to at least one pathogen in the biological fluid.

33. The method of any one of claims 18 to 32, wherein the substance further comprises thymic stromal lymphopoietin (TSLP) (e.g., wherein the TSLP enhances the ability of innate immune cells in the biological fluid (e.g., neutrophils) and the complement receptor to kill bacteria in the biological fluid).

34. The method of any one of claims 18 to 33, wherein the substance further comprises a drug or other agent that enhances the ability of innate immune cells in the biological fluid (e.g., neutrophils) and complement to kill bacteria in the biological fluid.

35. The method of any one of claims 18 to 34, wherein the contacting step comprises conducting the biological fluid of the subject through a filter (e.g., a membrane or other structure) comprising the high surface area polymer.

36. The method of any one of claims 18 to 35, further comprising returning the biological fluid to the subject following the contacting step.

37. A system for treating a biological fluid of a subject having a microbial disease (e.g., infection or other disease caused by a pathogenic organism, e.g., virus, bacteria, fungus, or protozoa), the system comprising:
a pump;
a tubing line fluidly connected to the pump; and
a treatment filter,
wherein the pump conducts the biological fluid from the subject to the treatment filter (e.g., and, in certain embodiments, back to the subject), and
wherein the treatment filter comprises a high surface area polymer having a substance immobilized thereupon or associated therewith, said substance comprising one or more complement receptors.

38. The system of claim 37, further comprising:
an air trap and/or air detector through which the tubing line passes prior to return of the biological fluid to the subject.

39. The system of claim 37 or 38, wherein the pump removes bubbles and/or microbubbles in the biological fluid before the biological fluid passes through the treatment filter.

40. The system of any one of claims 37 to 39, wherein the biological fluid is a member selected from the group consisting of blood, serum, plasma, cerebrospinal fluid, lymph, synovial fluid, and amniotic fluid.

41. The system of any one of claims 37 to 40, wherein the substance immobilized on the high surface area polymer comprises CRIg and a complement inhibitor selected from: sialic acid, Factor H protein (or a fragment thereof), a mini fH protein, or a fH-recruiting peptide.

42. The system of any one of claims 37 to 41, wherein the high surface area polymer has surface area of at least 50,000 cm²/g (e.g., more preferably, at least 100,000 cm²/g, more preferably, at least 150,000 cm²/g, or more preferably, at least 200,000 cm²/g).

43. The system of any one of claims 37 to 42, wherein the high surface area polymer is a membrane (e.g., hollow fiber membrane or other porous or nonporous membrane).

44. The system of any one of claims 37 to 43, wherein the high surface area polymer comprises particles having an average dimension (e.g., diameter) of less than 1 micrometer.

45. The system of any one of claims 37 to 44, wherein the high surface area polymer comprises poly(methyl methacrylate) (PMMA).

46. The system of any one of claims 37 to 45, wherein the high surface area polymer comprises nylon (e.g., Nylon-6, 6).

47. The system of any one of claims 37 to 46, wherein the high surface area polymer comprises one or more members selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof.

48. The system of any one of claims 37 to 47, wherein the biological fluid comprises methicillin-resistant Staphylococcus aureus (MRSA), and wherein the system enhances attachment of the one or more complement receptors to at least one pathogen in the biological fluid.

49. The system of any one of claims 37 to 48, wherein the biological fluid comprises one or more infectious microbes selected from the group consisting of Methicillin-Resistant Staphylococcus aureus, Streptococcus pneumoniae, Carbapenem-Resistant Enterobacteriaceae, Clostridium difficile, Drug-Resistant Neisseria gonorrhoeae, Drug-Resistant Malaria, and Multi-drug resistant (MDR) or extensively drug resistant (XDR) Tuberculosis, and wherein the system enhances the killing of the one or more infectious microbes.

50. The system of any one of claims 37 to 49, wherein the substance further comprises thymic stromal lymphopoietin (TSLP) (e.g., wherein the TSLP enhances the ability of innate immune cells in the biological fluid (e.g., neutrophils) and the complement to kill bacteria in the biological fluid).

51. A method of operating the system of any one of claims 37 to 50, the method comprising conducting a flow of the biological fluid over the surface of the high surface area polymer in the treatment filter, whereupon the polymer activates or enhances activation of the one or more complement receptors, thereby enhancing the in the biological fluid attachment of the one or more complement receptors onto at least one pathogen in the biological fluid.

52. A method for treating a biological fluid of a subject, the method comprising: contacting the biological fluid of the subject with particles (and/or fibers or other substrate) associated with a complement receptor, wherein the particles (and/or fibers or other substrate) bind microbes in the biological fluid.

53. A method for removing microbes from a biological fluid of a subject, the method comprising: contacting the biological fluid of the subject with particles (and/or fibers or other substrate) associated with a complement receptor, wherein the particles (and/or fibers or other substrate) bind microbes in the biological fluid.

54. The method of any one of claims 51 to 53, wherein the complement receptor is a CR1, CR2, CR3, CR4, CR3aR, CR5aR, C1qR, C1qRp, CRIg, or complement binding portion of any thereof.

55. The method of any one of claims 51 to 53, wherein the complement receptor is a CR1 or complement binding portion thereof and/or a CR2 or complement binding portion thereof.

56. The method of any one of claims 51 to 53, wherein the complement receptor is a CRIg or complement binding portion thereof.

57. The method of any one of claims 51 to 56, wherein the particles are further associated with a complement inhibitor selected from the group consisting of sialic acid, Factor H protein (or a fragment thereof), a mini fH protein, and a fH-recruiting peptide.

58. The method of claim 52 or 53, wherein the particles (and/or fibers or other substrate) associated with the complement receptor comprise a high surface area polymer selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof.

59. The method of claim 52 or 53, wherein the particles associated with the complement receptor comprise magnetic beads.

60. The method of any one of claim 52, 53, 58, or 59, wherein the particles are contained within a replaceable cartridge.

61. A system for treating a biological fluid of a subject, the system comprising:
a pump;
a tubing line fluidly connected to the pump; and
a cartridge,
wherein the pump conducts the biological fluid through the tubing line from the subject to the cartridge (e.g., and, in certain embodiments, back to the subject), and
wherein the cartridge comprises particles (and/or fibers or other substrate) associated with one or more complement receptors, wherein the particles (and/or fibers or other substrate) of the cartridge is/are capable of binding microbes in the biological fluid.

62. A system for removing microbes from a biological fluid of a subject, the system comprising: a pump and a tubing line for conducting the biological fluid from the subject to a cartridge (e.g., and, in certain embodiments, back to the subject); and
the cartridge comprising particles (and/or fibers or other substrate) associated with one or more complement receptors, wherein the particles (and/or fibers or other substrate) of the cartridge are capable of binding microbes in the biological fluid.

63. The system of claim 61 or 62, wherein the one or more complement receptors comprise at least one member selected from the group consisting of CR1, CR2, CR3, CR4, CR3aR, CR5aR, CRIg, C1qR, C1qRp, and a complement binding portion of any thereof.

64. The system of claim 61 or 62, wherein the one or more complement receptors comprises (i) a CR1 or complement binding portion thereof and/or (ii) a CR2 or complement binding portion thereof.

65. The method of claim 61 or 62, wherein the one or more complement receptors comprises CRIg or a complement binding portion thereof.

66. The method of any one of claims 61 to 65, wherein the particles are further associated with a complement inhibitor selected from the group consisting of sialic acid, Factor H protein (or a fragment thereof), a mini fH protein, or a fH-recruiting peptide.

67. The system of any one of claims 61 to 66, wherein the particles (and/or fibers or other substrate) associated with the one or more complement receptors comprise a high surface area polymer selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof.

68. The system of any one of claims 61 to 67, wherein the particles associated with the one or more complement receptors comprise magnetic beads.

69. The system of any one of claims 61 to 68, wherein the cartridge is a replaceable cartridge.

70. The system of any one of claims 61 to 69, wherein cells and/or other substances >5 μm, >4 μm, >3 μm, >2 μm or >1 μm in characteristic dimension (e.g., diameter or length) are unable to enter the cartridge (and/or a treatment portion thereof).

71. A system for treating a biological fluid of a subject, the system comprising:
at least one dialyzer;
a first tubing line for conducting the biological fluid from the subject to the at least one dialyzer; and
a second tubing line for conducting a dialysate to the at least one dialyzer,
wherein the dialysate comprises a soluble complement receptor.

72. The system of claim 71, wherein the soluble complement receptor comprises at least one of CR1, CR2, CR3, CR4, CR3aR, CR5aR, C1qR, C1qRp, CRIg, and a complement binding portion of any thereof.

73. The system of claim 71 or 72, wherein the dialysate further comprises at least one complement inhibitor selected from the group consisting of sialic acid, Factor H protein (or a fragment thereof), a mini fH protein, and a fH-recruiting peptide.

74. The system of any one of claims 71 to 73, wherein the dialyzer comprises at least one membrane (e.g., a membrane comprising a high surface area polymer).

75. The system of any one of claims 71 to 74, further comprising:
a dialysate supply tank fluidly connected to the second tubing line; and
a compressed air tank fluidly connected upstream of the dialysate supply tank.

76. The system of any one of claims 71 to 75, the dialyzer further comprising:
a dialysate inlet through which dialysate enters the dialyzer;
a dialysate exit, through which dialysate exits the dialyzer; and
at least one sensor disposed within at least one of the dialysate inlet and the dialysate exit.

77. The system of claim 76, wherein the at least one sensor comprises a spectrographic UV sensor.

78. The system of any one of claims 71 to 77, the dialyzer further comprising:
a blood inlet, through which the biological fluid enters the dialyzer;
a top header, the top header in fluid communication with and downstream of the blood inlet;
a blood exit, through which the biological fluid exits the dialyzer;
a bottom header, the bottom header in fluid communication with and upstream of the blood exit; and
at least one hollow fiber membrane disposed between the top header and the bottom header,
wherein the at one hollow fiber membrane fluidly connects the top header and the bottom header.

79. The system of any one of claims 71 to 75, the dialyzer further comprising:
at least one hollow fiber membrane.

80. The system of claim 79, wherein the at least one hollow fiber membrane further comprises a high surface area polymer.

81. The system of claim 80, wherein the high surface area polymer comprises one or more members selected from the group consisting of PMMA, nylon-6,6, poly(N-isopropyl acrylamide) (PIPA), poly(lactic acid) (PLLA), polyvinylchloride (PVC), polytetrafluoroethylene (PTFE), polyethersulfone (PES), polyethylene (PE), polyetheretherketone (PEEK), polysulfone (PS), polypropylene (PP), polyphenylsulfone (PPSU), polyoxymethylene (POM), and a copolymer thereof.

82. The system of any one of claims 71 to 81, wherein the biological fluid is a member selected from the group consisting of blood, serum, plasma, cerebrospinal fluid, lymph, synovial fluid, and amniotic fluid.

83. The system of claim 80, wherein the high surface area polymer has surface area of at least 50,000 cm²/g (e.g., more preferably, at least 100,000 cm²/g, more preferably, at least 150,000 cm²/g, or more preferably, at least 200,000 cm²/g).

84. The system of claim 79, wherein the at least one hollow fiber membrane has a cylindrical shape comprising an interior and an exterior, wherein the biological fluid flows through the interior of the hollow fiber membrane, and wherein the dialysate flows around the exterior of the hollow fiber membrane.

85. The system of claim 79 or 84, wherein the at least one hollow fiber membrane fluidly connects the at least one biological fluid to the dialysate.

86. The system of any one of claims 71 to 85, wherein the dialyzer comprises a membrane between the biological fluid and the dialysate, the membrane having a structure (e.g., pores) such that cells and/or other substances >5 μm, >4 μm, >3 μm, >2 μm, or >1 μm in a characteristic dimension (e.g., diameter or length) are unable to pass from the biological fluid into the dialysate (e.g., but pathogens can pass from the biological fluid into the dialysate).

87. A method for treating a biological fluid of a subject having a microbial disease (e.g., extracorporeal treatment), the method comprising:
providing a dialysate comprising at least one complement receptor on a first side of a membrane (e.g., a porous polymer membrane, e.g., a hollow fiber membrane); and
providing the biological fluid on a second side of the membrane, wherein the membrane is structured (e.g., has pores) to allow passage of a pathogen from the biological fluid through the membrane into the dialysate.

88. The method of claim 87, the method further comprising:
returning the biological fluid to the subject following a period of time in which components of the biological fluid are in fluid contact with the dialysate.

89. The method of claim 87 or 88, wherein the dialysate flows on the first side of the membrane.

90. The method of any one of claims 87 to 89, wherein the biological fluid flows on the second side of the membrane.

91. A method for treating a biological fluid of a subject having a microbial infection, the method comprising:
introducing a dialysate to a peritoneal cavity of the subject, wherein the dialysate comprises at least one complement receptor, such that the dialysate is in contact with the biological fluid; and
removing the dialysate from the biological fluid after sufficient time to opsonize a pathogen.

92. The method of claim 91, wherein the method is or includes peritoneal dialysis, such as continuous ambulatory peritoneal dialysis (CAPD), continuous cycling peritoneal dialysis (CCPD), and/or intermittent peritoneal dialysis (IPD).
